SEQUENCE LISTING (105 sequences)

SEQ ID NO: 1 - protein, 10 aa, Artificial Sequence
  source: /note=“Description of Artificial Sequence Synthetic peptide”
GFTLTDDYMT

SEQ ID NO: 2 - protein, 10 aa, Artificial Sequence
  source: /note=“Description of Artificial Sequence Synthetic peptide”
GGPFRSYAIS

SEQ ID NO: 3 - protein, 10 aa, Artificial Sequence
  source: /note=“Description of Artificial Sequence Synthetic peptide”
GFTFSTYAMH

SEQ ID NO: 4 - protein, 10 aa, Artificial Sequence
  source: /note=“Description of Artificial Sequence Synthetic peptide”
EVTFSSFAIS

SEQ ID NO: 5 - protein, 12 aa, Artificial Sequence
  source: /note=“Description of Artificial Sequence Synthetic peptide”
FIRDRANGYTTE

SEQ ID NO: 6 - protein, 10 aa, Artificial Sequence
  source: /note=“Description of Artificial Sequence Synthetic peptide”
GIIPIFGTTK

SEQ ID NO: 7 - protein, 9 aa, Artificial Sequence
  source: /note=“Description of Artificial Sequence Synthetic peptide”
VISYDANYK

SEQ ID NO: 8 - protein, 10 aa, Artificial Sequence
  source: /note=“Description of Artificial Sequence Synthetic peptide”
GISPMFGTPN

SEQ ID NO: 9 - protein, 11 aa, Artificial Sequence
  source: /note=“Description of Artificial Sequence Synthetic peptide”
PKGYFPYAMDY

SEQ ID NO: 10 - protein, 12 aa, Artificial Sequence
  source: /note=“Description of Artificial Sequence Synthetic peptide”
HMGYQVRETMDV

SEQ ID NO: 11 - protein, 20 aa, Artificial Sequence
  source: /note=“Description of Artificial Sequence Synthetic peptide”
DSQLRSLLYFEWLSQGYFDY

SEQ ID NO: 12 - protein, 15 aa, Artificial Sequence
  source: /note=“Description of Artificial Sequence Synthetic peptide”
SPSYICSGGTCVFDH

SEQ ID NO: 13 - protein, 11 aa, Artificial Sequence
  source: /note=“Description of Artificial Sequence Synthetic peptide”
LASQTIGTWLA

SEQ ID NO: 14 - protein, 13 aa, Artificial Sequence
  source: /note=“Description of Artificial Sequence Synthetic peptide”
SGSSSNIGNDYVS

SEQ ID NO: 15 - protein, 15 aa, Artificial Sequence
  source: /note=“Description of Artificial Sequence Synthetic peptide”
KSSQSVTFNYKNYLA

SEQ ID NO: 16 - protein, 13 aa, Artificial Sequence
  source: /note=“Description of Artificial Sequence Synthetic peptide”
TGNSNNVGNQGAA

SEQ ID NO: 17 - protein, 7 aa, Artificial Sequence
  source: /note=“Description of Artificial Sequence Synthetic peptide”
AATSLAD

SEQ ID NO: 18 - protein, 7 aa, Artificial Sequence
  source: /note=“Description of Artificial Sequence Synthetic peptide”
DNNKRPS

SEQ ID NO: 19 - protein, 7 aa, Artificial Sequence
  source: /note=“Description of Artificial Sequence Synthetic peptide”
WASTRES

SEQ ID NO: 20 - protein, 7 aa, Artificial Sequence
  source: /note=“Description of Artificial Sequence Synthetic peptide”
RNNDRPS

SEQ ID NO: 21 - protein, 9 aa, Artificial Sequence
  source: /note=“Description of Artificial Sequence Synthetic peptide”
QQLYSTPWT

SEQ ID NO: 22 - protein, 12 aa, Artificial Sequence
  source: /note=“Description of Artificial Sequence Synthetic peptide”
ATWDRRPTAYVV

SEQ ID NO: 23 - protein, 9 aa, Artificial Sequence
  source: /note=“Description of Artificial Sequence Synthetic peptide”
QQHYRTPPT

SEQ ID NO: 24 - protein, 11 aa, Artificial Sequence
  source: /note=“Description of Artificial Sequence Synthetic peptide”
STWDSSLSAVV

SEQ ID NO: 25 - protein, 122 aa, Artificial Sequence
  source: /note=“Description of Artificial Sequence Synthetic polypeptide”
EVKLVESGGGLVQPGGSLRLSCGTSGFTLTDDYMTWVRQPPGKALEWLGFIRDRANGYTTEYSASVKGRFTISRDNSQSIVYLQMNTLRVEDSATYYCARPKGYFPYAMDYWGQGTSVIVSS

SEQ ID NO: 26 - protein, 107 aa, Artificial Sequence
  source: /note=“Description of Artificial Sequence Synthetic polypeptide”
DIQMTQSPASQSASLGESVTITCLASQTIGTWLAWYQQKPGKSPQLLIYAATSLADGVPSRFSGSGSGTKFSFKISSLQAEDFVSYYCQQLYSTPWTFGGGTRLEIK

SEQ ID NO: 27 - protein, 121 aa, Artificial Sequence [sequence omitted]

SEQ ID NO: 28 - protein, 111 aa, Artificial Sequence [sequence omitted]

SEQ ID NO: 29 - protein, 124 aa, Artificial Sequence
  source: /note=“Description of Artificial Sequence Synthetic polypeptide”
QVQLVQSGAEVKKPGSSVKVSCTSSEVTFSSFAISWVRQAPGQGLEWLGGISPMFGTPNYAQKFQGRVTITADQSTRTAYMDLRSLRSEDTAVYYCARSPSYICSGGTCVFDHWGQGTLVTVSS

SEQ ID NO: 30 - protein, 121 aa, Artificial Sequence
  source: /note=“Description of Artificial Sequence Synthetic polypeptide”
IQPGLTQPPSVSKGLRQTATLTCTGNSNNVGNQGAAWLQQHQGHPPKLLSYRNNDRPSGISERFSASRSGNTASLTITGLQPEDEADYYCSTWDSSLSAVVFGGGTKLTVLGQPKAAPSAA

SEQ ID NO: 31 - protein, 129 aa, Artificial Sequence
  source: /note=“Description of Artificial Sequence Synthetic polypeptide”
QVQLVESGGGVVQPGRSLRLSCAASGFTFSTYAMHWVRQAPGKGLEWVAVISYDANYKYYADSVKGRFTISRDNSKNTLYLQMNSLRAEDTAVYYCAKDSQLRSLLYFEWLSQGYFDYWGQGTLVTVSS

SEQ ID NO: 32 - protein, 111 aa, Artificial Sequence
  source: /note=“Description of Artificial Sequence Synthetic polypeptide”
DIVMTQSPDSLAVSLGERATINCKSSQSVTFNYKNYLAWYQQKPGQPPKLLIYWASTRESGVPDRFSGSGSGTDFTLTISSLQAEDVAVYYCQQHYRTPPTFGQGTKVEIK

SEQ ID NO: 33 - protein, 10 aa, Artificial Sequence
  source: /note=“Description of Artificial Sequence Synthetic peptide”
GYTFTDYHIN

SEQ ID NO: 34 - protein, 11 aa, Artificial Sequence
  source: /note=“Description of Artificial Sequence Synthetic peptide”
GYSISSNYYWG

SEQ ID NO: 35 - protein, 10 aa, Artificial Sequence
  source: /note=“Description of Artificial Sequence Synthetic peptide”
EFNFKSYWMT

SEQ ID NO: 36 - protein, 10 aa, Artificial Sequence
  source: /note=“Description of Artificial Sequence Synthetic peptide”
WIHPNSGDTN

SEQ ID NO: 37 - protein, 9 aa, Artificial Sequence
  source: /note=“Description of Artificial Sequence Synthetic peptide”
SIYHSGSTY

SEQ ID NO: 38 - protein, 10 aa, Artificial Sequence
  source: /note=“Description of Artificial Sequence Synthetic peptide”
NINQDGSEKN

SEQ ID NO: 39 - protein, 17 aa, Artificial Sequence
  source: /note=“Description of Artificial Sequence Synthetic peptide”
GGLEPRSVDYYYYGMDV

SEQ ID NO: 40 - protein, 15 aa, Artificial Sequence
  source: /note=“Description of Artificial Sequence Synthetic peptide”
HVRSGYPDTAYYFDK

SEQ ID NO: 41 - protein, 16 aa, Artificial Sequence
  source: /note=“Description of Artificial Sequence Synthetic peptide”
TGSSWDTYYYYYAMDV

SEQ ID NO: 42 - protein, 11 aa, Artificial Sequence
  source: /note=“Description of Artificial Sequence Synthetic peptide”
GGNDIGRKSVH

SEQ ID NO: 43 - protein, 11 aa, Artificial Sequence
  source: /note=“Description of Artificial Sequence Synthetic peptide”
GGNNIGTKVLH

SEQ ID NO: 44 - protein, 12 aa, Artificial Sequence
  source: /note=“Description of Artificial Sequence Synthetic peptide”
RASQSVSSSYLV

SEQ ID NO: 45 - protein, 7 aa, Artificial Sequence
  source: /note=“Description of Artificial Sequence Synthetic peptide”
YDSDRPS

SEQ ID NO: 46 - protein, 7 aa, Artificial Sequence
  source: /note=“Description of Artificial Sequence Synthetic peptide”
DDSDRPS

SEQ ID NO: 47 - protein, 7 aa, Artificial Sequence
  source: /note=“Description of Artificial Sequence Synthetic peptide”
GASSRAP

SEQ ID NO: 48 - protein, 11 aa, Artificial Sequence
  source: /note=“Description of Artificial Sequence Synthetic peptide”
QVWDSSSDHVI

SEQ ID NO: 49 - protein, 11 aa, Artificial Sequence
  source: /note=“Description of Artificial Sequence Synthetic peptide”
QVWDISTDQAV

SEQ ID NO: 50 - protein, 9 aa, Artificial Sequence
  source: /note=“Description of Artificial Sequence Synthetic peptide”
QQYGRSFGQ

SEQ ID NO: 51 - protein, 126 aa, Artificial Sequence
  source: /note=“Description of Artificial Sequence Synthetic polypeptide”
EVQLVQSGAEVKKPGASVKVSCKASGYTFTDYHINWVRQAPGQGLEWMGWIHPNSGDTNYAQKFQGWVTMTRDTAISTAYMEVNGLKSDDTAVYYCARGGLEPRSVDYYYYGMDVWGQGTTVTVSS

SEQ ID NO: 52 - protein, 108 aa, Artificial Sequence
  source: /note=“Description of Artificial Sequence Synthetic polypeptide”
QSVLTQPPSVSVAPGQTARITCGGNDIGRKSVHWNQQKPGQAPVLVVCYDSDRPSGIPERFSGSNSGNTATLTISRVEAGDEADYYCQVWDSSSDHVIFGGGTKLTVL

SEQ ID NO: 53 - protein, 124 aa, Artificial Sequence
  source: /note=“Description of Artificial Sequence Synthetic polypeptide”
EVQLVESGPGLVKPSDILSLTCAVSGYSISSNYYWGWIRQPPGKGLEWIGSIYHSGSTYYKPSLESRLGISVDTSKNQFSLKLSFVSAADTAVYYCARHVRSGYPDTAYYFDKWGQGTLVTVSS

SEQ ID NO: 54 - protein, 109 aa, Artificial Sequence
  source: /note=“Description of Artificial Sequence Synthetic polypeptide”
TSYVLTQPPSVSVAPGETARISCGGNNIGTKVLHWYQQTPGQAPVLVVYDDSDRPSGIPERFSGSNSGNTATLTISRVEVGDEADYYCQVWDISTDQAVFGGGTKLTVL

SEQ ID NO: 55 - protein, 125 aa, Artificial Sequence
  source: /note=“Description of Artificial Sequence Synthetic polypeptide”
EVQLVESGGGLVQPGGSLRLSCAASEFNFKSYWMTWVRQAPGKGLEWVANINQDGSEKNYVDSVKGRFTISRDNAKNSLHLQMSSLRVDDTAVYYCARTGSSWDTYYYYYAMDVWGQGTTVTVSS

SEQ ID NO: 56 - protein, 105 aa, Artificial Sequence
  source: /note=“Description of Artificial Sequence Synthetic polypeptide”
DIQLTQSPVSLSLSPGERATLSCRASQSVSSSYLVWYQQKPGQAPRLLIYGASSRAPGIPDRFSGSGSGTDFTLTISRLEREDFAVYYCQQYGRSFGQGTKVEIK

SEQ ID NO: 57 - protein, 25 aa, Artificial Sequence
  source: /note=“Description of Artificial Sequence Synthetic peptide”
EVQLVQSGAEVKKPGASVKVSCKAS

SEQ ID NO: 58 - protein, 14 aa, Artificial Sequence
  source: /note=“Description of Artificial Sequence Synthetic peptide”
WVRQAPGQGLEWMG

SEQ ID NO: 59 - protein, 39 aa, Artificial Sequence
  source: /note=“Description of Artificial Sequence Synthetic polypeptide”
YAQKFQGWVTMTRDTAISTAYMEVNGLKSDDTAVYYCAR

SEQ ID NO: 60 - protein, 11 aa, Artificial Sequence
  source: /note=“Description of Artificial Sequence Synthetic peptide”
WGQGTTVTVSS

SEQ ID NO: 61 - protein, 22 aa, Artificial Sequence
  source: /note=“Description of Artificial Sequence Synthetic peptide”
QSVLTQPPSVSVAPGQTARITC

SEQ ID NO: 62 - protein, 15 aa, Artificial Sequence
  source: /note=“Description of Artificial Sequence Synthetic peptide”
WNQQKPGQAPVLVVC

SEQ ID NO: 63 - protein, 32 aa, Artificial Sequence
  source: /note=“Description of Artificial Sequence Synthetic polypeptide”
GIPERFSGSNSGNTATLTISRVEAGDEADYYC

SEQ ID NO: 64 - protein, 10 aa, Artificial Sequence
  source: /note=“Description of Artificial Sequence Synthetic peptide”
FGGGTKLTVL

SEQ ID NO: 65 - protein, 25 aa, Artificial Sequence
  source: /note=“Description of Artificial Sequence Synthetic peptide”
EVQLVESGPGLVKPSDILSLTCAVS

SEQ ID NO: 66 - protein, 14 aa, Artificial Sequence
  source: /note=“Description of Artificial Sequence Synthetic peptide”
WIRQPPGKGLEWIG

SEQ ID NO: 67 - protein, 39 aa, Artificial Sequence
  source: /note=“Description of Artificial Sequence Synthetic polypeptide”
YKPSLESRLGISVDTSKNQFSLKLSFVSAADTAVYYCAR

SEQ ID NO: 68 - protein, 11 aa, Artificial Sequence
  source: /note=“Description of Artificial Sequence Synthetic peptide”
WGQGTLVTVSS

SEQ ID NO: 69 - protein, 23 aa, Artificial Sequence
  source: /note=“Description of Artificial Sequence Synthetic peptide”
TSYVLTQPPSVSVAPGETARISC

SEQ ID NO: 70 - protein, 15 aa, Artificial Sequence
  source: /note=“Description of Artificial Sequence Synthetic peptide”
WYQQTPGQAPVLVVY

SEQ ID NO: 71 - protein, 32 aa, Artificial Sequence
  source: /note=“Description of Artificial Sequence Synthetic polypeptide”
GIPERFSGSNSGNTATLTISRVEVGDEADYYC

SEQ ID NO: 72 - protein, 25 aa, Artificial Sequence [sequence omitted]

SEQ ID NO: 73 - protein, 14 aa, Artificial Sequence
  source: /note=“Description of Artificial Sequence Synthetic peptide”
WVRQAPGKGLEWVA

SEQ ID NO: 74 - protein, 39 aa, Artificial Sequence
  source: /note=“Description of Artificial Sequence Synthetic polypeptide”
YVDSVKGRFTISRDNAKNSLHLQMSSLRVDDTAVYYCAR

SEQ ID NO: 75 - protein, 23 aa, Artificial Sequence
  source: /note=“Description of Artificial Sequence Synthetic peptide”
DIQLTQSPVSLSLSPGERATLSC

SEQ ID NO: 76 - protein, 15 aa, Artificial Sequence
  source: /note=“Description of Artificial Sequence Synthetic peptide”
WYQQKPGQAPRLLIY

SEQ ID NO: 77 - protein, 32 aa, Artificial Sequence
  source: /note=“Description of Artificial Sequence Synthetic polypeptide”
GIPDRFSGSGSGTDFTLTISRLEREDFAVYYC

SEQ ID NO: 78 - protein, 7 aa, Artificial Sequence
  source: /note=“Description of Artificial Sequence Synthetic peptide”
GTKVEIK

SEQ ID NO: 79 - protein, 25 aa, Artificial Sequence
  source: /note=“Description of Artificial Sequence Synthetic peptide”
EVKLVESGGGLVQPGGSLRLSCGTS

SEQ ID NO: 80 - protein, 14 aa, Artificial Sequence
  source: /note=“Description of Artificial Sequence Synthetic peptide”
WVRQPPGKALEWLG

SEQ ID NO: 81 - protein, 39 aa, Artificial Sequence
  source: /note=“Description of Artificial Sequence Synthetic polypeptide”
YSASVKGRFTISRDNSQSIVYLQMNTLRVEDSATYYCAR

SEQ ID NO: 82 - protein, 11 aa, Artificial Sequence
  source: /note=“Description of Artificial Sequence Synthetic peptide”
WGQGTSVIVSS

SEQ ID NO: 83 - protein, 23 aa, Artificial Sequence
  source: /note=“Description of Artificial Sequence Synthetic peptide”
DIQMTQSPASQSASLGESVTITC

SEQ ID NO: 84 - protein, 15 aa, Artificial Sequence
  source: /note=“Description of Artificial Sequence Synthetic peptide”
WYQQKPGKSPQLLIY

SEQ ID NO: 85 - protein, 32 aa, Artificial Sequence
  source: /note=“Description of Artificial Sequence Synthetic polypeptide”
GVPSRFSGSGSGTKFSFKISSLQAEDFVSYYC

SEQ ID NO: 86 - protein, 10 aa, Artificial Sequence
  source: /note=“Description of Artificial Sequence Synthetic peptide”
FGGGTRLEIK

SEQ ID NO: 87 - protein, 25 aa, Artificial Sequence
  source: /note=“Description of Artificial Sequence Synthetic peptide”
EVQLVESGAEVKKPGSSVKVSCKAS

SEQ ID NO: 88 - protein, 14 aa, Artificial Sequence
  source: /note=“Description of Artificial Sequence Synthetic peptide”
WVRQAPGQGPEWMG

SEQ ID NO: 89 - protein, 39 aa, Artificial Sequence
  source: /note=“Description of Artificial Sequence Synthetic polypeptide”
YAPKFQGRVTITADDFAGTVYMELSSLRSEDTAMYYCAK

SEQ ID NO: 90 - protein, 11 aa, Artificial Sequence
  source: /note=“Description of Artificial Sequence Synthetic peptide”
WGKGTTVTVSS

SEQ ID NO: 91 - protein, 22 aa, Artificial Sequence
  source: /note=“Description of Artificial Sequence Synthetic peptide”
QSVLTQPPSVSAAPGQKVTISC

SEQ ID NO: 92 - protein, 15 aa, Artificial Sequence
  source: /note=“Description of Artificial Sequence Synthetic peptide”
WYQQLPGTAPKLLIY

SEQ ID NO: 93 - protein, 32 aa, Artificial Sequence
  source: /note=“Description of Artificial Sequence Synthetic polypeptide”
GIPDRFSGSKSGTSATLGITGLQTGDEANYYC

SEQ ID NO: 94 - protein, 25 aa, Artificial Sequence
  source: /note=“Description of Artificial Sequence Synthetic peptide”
QVQLVESGGGVVQPGRSLRLSCAAS

SEQ ID NO: 95 - protein, 40 aa, Artificial Sequence
  source: /note=“Description of Artificial Sequence Synthetic polypeptide”
YYADSVKGRFTISRDNSKNTLYLQMNSLRAEDTAVYYCAK

SEQ ID NO: 96 - protein, 23 aa, Artificial Sequence
  source: /note=“Description of Artificial Sequence Synthetic peptide”
DIVMTQSPDSLAVSLGERATINC

SEQ ID NO: 97 - protein, 15 aa, Artificial Sequence
  source: /note=“Description of Artificial Sequence Synthetic peptide”
WYQQKPGQPPKLLIY

SEQ ID NO: 98 - protein, 32 aa, Artificial Sequence
  source: /note=“Description of Artificial Sequence Synthetic polypeptide”
GVPDRFSGSGSGTDFTLTISSLQAEDVAVYYC

SEQ ID NO: 99 - protein, 10 aa, Artificial Sequence
  source: /note=“Description of Artificial Sequence Synthetic peptide”
FGQGTKVEIK

SEQ ID NO: 100 - protein, 25 aa, Artificial Sequence
  source: /note=“Description of Artificial Sequence Synthetic peptide”
QVQLVQSGAEVKKPGSSVKVSCTSS

SEQ ID NO: 101 - protein, 14 aa, Artificial Sequence
  source: /note=“Description of Artificial Sequence Synthetic peptide”
WVRQAPGQGLEWLG

SEQ ID NO: 102 - protein, 39 aa, Artificial Sequence
  source: /note=“Description of Artificial Sequence Synthetic polypeptide”
YAQKFQGRVTITADQSTRTAYMDLRSLRSEDTAVYYCAR

SEQ ID NO: 103 - protein, 23 aa, Artificial Sequence
  source: /note=“Description of Artificial Sequence Synthetic peptide”
IQPGLTQPPSVSKGLRQTATLTC

SEQ ID NO: 104 - protein, 15 aa, Artificial Sequence
  source: /note=“Description of Artificial Sequence Synthetic peptide”
WLQQHQGHPPKLLSY

SEQ ID NO: 105 - protein, 32 aa, Artificial Sequence
  source: /note=“Description of Artificial Sequence Synthetic polypeptide”
GISERFSASRSGNTASLTITGLQPEDEADYYC

(19) United States
(12) Patent Application Publication
(10) Pub. No.: US 2017/0121373 A1
(43) Pub. Date: May 4, 2017

(54) EXPRESSION AND CONFORMATIONAL ANALYSIS OF ENGINEERED INFLUENZA HEMAGGLUTININ

(71) Applicant: SANOFI PASTEUR INC., Swiftwater, PA (US)

(72) Inventors: Guadalupe CORTES-GARCIA, Swiftwater, PA (US); Timothy ALEFANTIS, Swiftwater, PA (US); Mark PARRINGTON, Swiftwater, PA (US)

(21) Appl. No.: 15/315,257

(22) PCT Filed: May 29, 2015
(86) PCT No.: PCT/US15/33205

§ 371 Date: Nov. 30, 2016

Related U.S. Application Data
(60) Provisional application No. 62/005,728, filed on May 30, 2014.

(51) Int. Cl. C07K 14/005; A61K 39/145; G01N 33/569

(52) U.S. Cl. CPC C07K 14/005; G01N 33/56983; A61K 39/145; G01N 2333/11; C12N 2760/16022; C12N 2760/16034; A61K 2039/57

(57) ABSTRACT

The present invention provides, among other things, compositions and methods for analyzing the expression and conformation of engineered influenza hemagglutinin In particular, the present invention provides methods of screening in silico designed HA antigens using neutralizing antibody panels specific to conserved epitopes. In some embodiments, the HAs are down selected for inclusion in universal influenza vaccines based upon their binding to neutralizing antibody panels in immunostaining assays.

### CROSS REFERENCE TO RELATED APPLICATIONS

This application claims priority to U.S. Provisional Application 62/005,728 filed on May 30, 2014, which is incorporated by reference in its entirety

### BACKGROUND

Influenza has a long standing history of pandemics, epidemics, resurgences and outbreaks. Vaccines have been the most effective defense against influenza. Most of the currently marketed influenza vaccines are based on inducing immunity to the hemagglutinin (HA) antigen present on the surface of influenza viruses. Hemagglutinin (HA) is a glycoprotein responsible for the binding of the influenza virus to cells with sialic acid on the membranes, and is highly variable across influenza virus strains. Among the current strategies for effective vaccination against influenza, the development of universal vaccines with increased breadth of immunity based on conserved and/or cross-reactive epitopes holds the promise of addressing the limitations of current strain-specific seasonal vaccines. Systematic bioinformatics analysis of HA sequences provides the means for engineering or re-engineering HA antigens to incorporate such cross-reactive epitopes; however it also creates the need for robust in vitro screening assays to identify most promising in silico designs for pre-clinical or clinical studies.

### SUMMARY

The present invention provides a robust and rapid screening assay to identify promising designs that produce functional influenza hemagglutinin (HA) antigens for universal vaccines. The present invention is, in part, based on the successful development of a flow cytometry based assay that utilizes a panel of neutralizing antibodies to analyze expression and conformation of surface displayed engineered HA antigens. As demonstrated in the Examples section, inventive assays provided by the present invention not only identify and validate engineered HA antigens that are properly expressed and structurally sound, but also predict the breath and/or specificity of immunogenicity of engineered HA antigens.

In one aspect, the present invention provides methods of analyzing expression and conformation of engineered hemagglutinin (HA) antigens, comprising steps of (a) providing one or more cells, each cell comprising a nucleic acid sequence encoding an engineered HA antigen; (b) immunostaining of the one or more cells with a panel of neutralizing antibodies under conditions that permit the neutralizing antibodies to bind to the engineered HA antigen displayed on surface of the one or more cells, wherein the panel of neutralizing antibodies comprise a plurality of neutralizing antibodies against HA stem and a plurality of neutralizing antibodies against HA head; (c) detecting binding levels between individual neutralizing antibodies and the engineered HA antigen displayed on the surface of the one or more cells; and (d) determining if the engineered HA antigen is properly expressed and/or folded based on the binding levels detected between the individual neutralizing antibodies and the engineered HA antigen.

In some embodiments, the nucleic acid sequence encoding an engineered HA antigen is a plasmid sequence.

In some embodiments, the engineered HA antigen is designed by computational approaches. In some embodiments, the engineered HA antigen is designed based on consensus sequences among a series of HA proteins from different influenza strains. In some embodiments, the engineered HA antigen is designed based on the deletion or rearrangement of structural domains. In some embodiments, the engineered HA antigen is designed based on swap of structural domains derived from multiple influenza strains. In some embodiments, the engineered HA antigen is rationally designed based on combinations of neutralizing, hemagglutinin B-cell epitope patterns derived from multiple influenza strains. In some embodiments, the engineered HA antigen comprises cross-reactive epitopes.

In some embodiments, the panel of neutralizing antibodies comprise at least three neutralizing antibodies against HA stem and at least three neutralizing antibodies against HA head. In some embodiments, the plurality of neutralizing antibodies against HA stem comprise antibodies that bind specifically to one or more conserved epitopes in the stem of HA from multiple influenza strains. In some embodiments, the one or more conserved epitopes in the stem are within a region corresponding to HA2 A helix. In some embodiments, the one or more conserved epitopes of the stem region are defined by residues corresponding to HAI residues 18, 38, 40, 42, 291-293, and 318 or a subset thereof; HA2 residues 18-21, 38, 41-43, 45, 46, 49, 52, and 56 or a subset thereof; and/or H5 residues HA2 αA, 52, 53, and 56, or a subset thereof.

In some embodiments, the plurality of neutralizing antibodies against HA stem comprise antibodies defined by: a heavy chain CDR1 sequence selected from the group consisting of GFTLTDDYMT, GGPFRSYAIS, GFTFSTYAMH, and EVTFSSFAIS; a heavy chain CDR2 sequence selected from the group consisting of FIRDRANGYTTE, GIIPIFGTTK, VISYDANYK, and GISPMFGTPN; a heavy chain CDR3 sequence selected from the group consisting of PKGYFPYAMDY, HMGYQVRETMDV, DSQLRSLLYFEWLSQGYFDY and SPSYICSGGTCVFDH; a light chain CDR1 sequence selected from the group consisting of LASQTIGTWLA, SGSSSNIGNDYVS, KSSQSVTFNYKNYLA and TGNSNNVGNQGAA; a light chain CDR2 sequence selected from the group consisting of AATSLAD, DNNKRPS, WASTRES and RNNDRPS; or a light chain CDR3 sequence selected from the group consisting of QQLYSTPWT, ATWDRRPTAYVV, QQHYRTPPT and STWDSSLSAVV.

In some embodiments, the plurality of neutralizing antibodies against HA stem comprise antibodies selected from the group consisting of:
antibody comprising a heavy chain comprising a CDR1 sequence of GFTLTDDYMT, CDR2 sequence of FIRDRANGYTTE, and CDR3 sequence of PKGYFPYAMDY; and a light chain comprising a CDR1 sequence of LASQTIGTWLA, CDR2 sequence of AATSLAD, and CDR3 sequence of QQLYSTPWT;
antibody comprising a heavy chain comprising a CDR1 sequence of GGPFRSYAIS, CDR2 sequence of GIIPIFGTTK, and CDR3 sequence of HMGYQVRETMDV; and a light chain comprising a CDR1 sequence of SGSSSNIGNDYVS, CDR2 sequence of DNNKRPS, and CDR3 sequence of ATWDRRPTAYVV;
antibody comprising a heavy chain comprising a CDR1 sequence of EVTFSSFAIS, CDR2 sequence of GISPMFGTPN, and CDR3 sequence of SPSYICSGGTCVFDH; and a light chain comprising a CDR1 sequence of TGNSNNVGNQGAA, CDR2 sequence of RNNDRPS, and CDR3 sequence of STWDSSLSAVV;
antibody comprising a heavy chain comprising a CDR1 sequence of GFTFSTYAMH, CDR2 sequence of VISYDANYK, and CDR3 sequence of DSQLRSLLYFEWLSQGYFDY; and a light chain comprising a CDR1 sequence of KSSQSVTFNYKNYLA, CDR2 sequence of WASTRES, and CDR3 sequence of QQHYRTPPT; and combination thereof.

In some embodiments, the plurality of neutralizing antibodies against HA stem comprise antibodies selected from the group consisting of:
antibody comprising a heavy chain having an amino acid sequence at least 90% (e.g., at least 92%, 94%, 96%, 98%, or 99%) identical to EVKLVESGGGLVQPGGSLRLSCGTSGFTLTDDYMTWVRQPPGKALEWLGFIRDRANGYTTEYSASVKGRFTI SRDNSQSIVYLQMNTLRVEDSATYYCARPKGYFPYAMDYWGQGTSVIVSS; and a light chain having an amino acid sequence at least 90% identical to DIQMTQSPASQSASLGESVTITCLASQTIGTWLAWYQQKPGKSPQLLIYAATSLADGVPSRFSGSGSGTKFSFKISSLQAEDFVSYYCQQLYSTPWTFGGGTRLEIK;
antibody comprising a heavy chain having an amino acid sequence at least 90% (e.g., at least 92%, 94%, 96%, 98%, or 99%) identical to EVQLVESGAEVKKPGSSVKVSCKASGGPFRSYAISWVRQAPGQGPEWMGGIIPIFGTTKYAPKFQGRVTITADDFAGTVYMELSSLRSEDTAMYYCAKHMGYQVRETMDVWGKGTTVTVSS; and a light chain having an amino acid sequence at least 90% (e.g., at least 92%, 94%, 96%, 98%, or 99%) identical to QSVLTQPPSVSAAPGQKVTISCSGSSSNIGNDYVSWYQQLPGTAPKLLIYDNNKRPSGIPDRFSGSKSGTSATLGITGLQTGDEANYYCATWDRRPTAYVVFGGGTKLTVL;
antibody comprising a heavy chain having an amino acid sequence at least 90% (e.g., at least 92%, 94%, 96%, 98%, or 99%) identical to QVQLVQSGAEVKKPGSSVKVSCTSSEVTFSSFAISWVRQAPGQGLEWLGGISPMFGTPNYAQKFQGRVTITADQSTRTAYMDLRSLRSEDTAVYYCARSPSYICSGGTCVFDHWGQGTLVTVSS; and a light chain having an amino acid sequence at least 90% (e.g., at least 92%, 94%, 96%, 98%, or 99%) identical to IQPGLTQPPSVSKGLRQTATLTCTGNSNNVGNQGAAWLQQHQGHPPKLLSYRNNDRPSGISERFSASRSGNTASLTITGLQPEDEADYYCSTWDSSLSAVVFGGGTKLTVLGQPKAAPSAA; antibody comprising a heavy chain having an amino acid sequence at least 90% (e.g., at least 92%, 94%, 96%, 98%, or 99%) identical to QVQLVESGGGVVQPGRSLRLSCAASGFTFSTYAMHWVRQAPGKGLEWVAVISYDANYKYYADSVKGRFTISRDNSKNTLYLQMNSLRAEDTAVYYCAKDSQLRSLLYFEWLSQGYFDYWGQGTLVTVSS; and a light chain having an amino acid sequence at least 90% (e.g., at least 92%, 94%, 96%, 98%, or 99%) identical to DIVMTQSPDSLAVSLGERATINCKSSQSVTFNYKNYLAWYQQKPGQPPKLLIYWASTRESGVPDRFSGSGSGTDFTLTISSLQAEDVAVYYCQQHYRTPPTFGQGTKVEIK; and combination thereof.

In some embodiments, the plurality of neutralizing antibodies against HA stem comprise antibodies selected from the group consisting of:
antibody comprising a heavy chain having an amino acid sequence of EVKLVESGGGLVQPGGSLRLSCGTSGFTLTDDYMTWVRQPPGKALEWLGFIRDRANGYTTEYSASVKGRFTI SRDNSQSIVYLQMNTLRVEDSATYYCARPKGYFPYAMDYWGQGTSVIVSS; and a light chain having an amino acid sequence of DIQMTQSPASQSASLGESVTITCLASQTIGTWLAWYQQKPGKSPQLLIYAATSLADGVPSRFSGSGSGTKFSFKISSLQAEDFVSYYCQQLYSTPWTFGGGTRLEIK;
antibody comprising a heavy chain having an amino acid sequence of EVQLVESGAEVKKPGSSVKVSCKASGGPFRSYAISWVRQAPGQGPEWMGGIIPIFGTTKYAPKFQGRVTITADDFAGTVYMELSSLRSEDTAMYYCAKHMGYQVRETMDVWGKGTTVTVSS; and a light chain having an amino acid sequence of QSVLTQPPSVSAAPGQKVTISCSGSSSNIGNDYVSWYQQLPGTAPKLLIYDNNKRPSGIPDRFSGSKSGTSATLGITGLQTGDEANYYCATWDRRPTAYVVFGGGTKLTVL;
antibody comprising a heavy chain having an amino acid sequence of QVQLVQSGAEVKKPGSSVKVSCTSSEVTFSSFAISWVRQAPGQGLEWLGGISPMFGTPNYAQKFQGRVTITADQSTRTAYMDLRSLRSEDTAVYYCARSPSYICSGGTCVFDHWGQGTLVTVSS; and a light chain having an amino acid sequence of IQPGLTQPPSVSKGLRQTATLTCTGNSNNVGNQGAAWLQQHQGHPPKLLSYRNNDRPS GISERFSASRSGNTASLTITGLQPEDEADYYCSTWDSSLSAVVFGGGTKLTVLGQPKAAPSAA;
antibody comprising a heavy chain having an amino acid sequence of QVQLVESGGGVVQPGRSLRLSCAASGFTFSTYAMHWVRQAPGKGLEWVAVISYDANYKYYADSVKGRFTISRDNSKNTLYLQMNSLRAEDTAVYYCAKDSQLRSLLYFEWLSQGYFDYWGQGTLVTVSS; and a light chain having an amino acid sequence of DIVMTQSPDSLAVSLGERATINCKSSQSVTFNYKNYLAWYQQKPGQPPKLLIYWASTRESGVPDRFSGSGSGTDFTLTISSLQAEDVAVYYCQQHYRTPPTFGQGTKVEIK; and combination thereof.

In some embodiments, the plurality of neutralizing antibodies against HA stem comprise antibodies that compete with one or more antibodies described herein (e.g., monoclonal antibodies FI6, C179, CR6261, and/or F10).

In some embodiments, the plurality of neutralizing antibodies against HA head comprise antibodies bind specifically to epitopes close to the receptor-binding site. In some embodiments, the epitopes close to the receptor-binding site correspond to the N-terminal end of the short a-helix, site Sa, site Sb, the edge of the receptor pocket, the C-terminus of the short α-helix. In some embodiments, the epitopes close to the receptor-binding site comprises an epitope defined by residues corresponding to H1N1 HA residues 133A, 137 and 222.

In some embodiments, the plurality of neutralizing antibodies against HA head comprise antibodies defined by: a heavy chain CDR1 sequence selected from the group consisting of GFTFSTYAMH, GYTFTDYHIN, GYSISSNYYWG, and EFNFKSYWMT; a heavy chain CDR2 sequence selected from the group consisting of VISYDANYK, WIHPNSGDTN, SIYHSGSTY, and NINQDGSEKN; a heavy chain CDR3 sequence selected from the group consisting of DSQLRSLLYFEWLSQGYFDY, GGLEPRSVDYYYYGMDV, HVRSGYPDTAYYFDK and TGSSWDTYYYYYAMDV; a light chain CDR1 sequence selected from the group consisting of KSSQSVTFNYKNYLA, GGNDIGRKSVH, GGNNIGTKVLH, and RASQSVSSSYLV; a light chain CDR2 sequence selected from the group consisting of WASTRES, YDSDRPS, DDSDRPS, and GASSRAP; and/or a light chain CDR3 sequence selected from the group consisting of QQHYRTPPT, QVWDSSSDHVI, QVWDISTDQAV, and QQYGRSFGQ.

In some embodiments, the plurality of neutralizing antibodies against HA head comprise antibodies selected from the group consisting of:
antibody comprising a heavy chain comprising a CDR1 sequence of GYTFTDYHIN, CDR2 sequence of WIHPNSGDTN, and CDR3 sequence of GGLEPRSVDYYYYGMDV; and a light chain comprising a CDR1 sequence of GGNDIGRKSVH, CDR2 sequence of YDSDRPS, and CDR3 sequence of QVWDSSSDHVI;
antibody comprising a heavy chain comprising a CDR1 sequence of GYSISSNYYWG, CDR2 sequence of SIYHSGSTY, and CDR3 sequence of HVRSGYPDTAYYFDK; and a light chain comprising a CDR1 sequence of GGNNIGTKVLH, CDR2 sequence of DDSDRPS, and CDR3 sequence of QVWDISTDQAV;
antibody comprising a heavy chain comprising a CDR1 sequence of EFNFKSYWMT, CDR2 sequence of NINQDGSEKN, and CDR3 sequence of TGSSWDTYYYYYAMDV; and a light chain comprising a CDR1 sequence of RASQSVSSSYLV, CDR2 sequence of GASSRAP, and CDR3 sequence of QQYGRSFGQ; and combination thereof.

In some embodiments, the plurality of neutralizing antibodies against HA head comprise antibodies selected from the group consisting of:
antibody comprising a heavy chain having an amino acid sequence at least 90% (e.g., at least 92%, 94%, 96%, 98%, or 99%) identical to EVQLVQSGAEVKKPGASVKVSCKASGYTFTDYHINWVRQAPGQGLEWMGWIHPNSGDTNYAQKFQGWVTMTRDTAISTAYMEVNGLKSDDTAVYYCARGGLEPRSVDYYYYGMDVWGQGTTVTVSS; and a light chain having an amino acid sequence at least 90% (e.g., at least 92%, 94%, 96%, 98%, or 99%) identical to QSVLTQPPSVSVAPGQTARITCGGNDIGRKSVHWNQQKPGQAPVLVVCYDSDRPSGIPERFSGSNSGNTATLTISRVEAGDEADYYCQVWDSSSDHVIFGGGTKLTVL;
antibody comprising a heavy chain having an amino acid sequence at least 90% (e.g., at least 92%, 94%, 96%, 98%, or 99%) identical to EVQLVESGPGLVKPSDILSLTCAVSGYSISSNYYWGWIRQPPGKGLEWIGSIYHSGSTYYKPSLESRLGISVDTSKNQFSLKLSFVSAADTAVYYCARHVRSGYPDTAYYFDKWGQGTLVTVSs; and a light chain having an amino acid sequence at least 90% (e.g., at least 92%, 94%, 96%, 98%, or 99%) identical to TSYVLTQPPSVSVAPGETARISCGGNNIGTKVLHWYQQTPGQAPVLVVYDDSDRPSGIPERFSGSNSGNTATLTISRVEVGDEADYYCQVWDISTDQAVFGGGTKLTVL;
antibody comprising a heavy chain having an amino acid sequence at least 90% (e.g., at least 92%, 94%, 96%, 98%, or 99%) identical to EVQLVESGGGLVQPGGSLRLSCAASEFNFKSYWMTWVRQAPGKGLEWVANINQDGSEKNYVDSVKGRFTISRDNAKNSLHLQMSSLRVDDTAVYYCARTGSSWDTYYYYYAMDVWGQGTTVTVSS; and a light chain having an amino acid sequence at least 90% (e.g., at least 92%, 94%, 96%, 98%, or 99%) identical to DIQLTQSPVSLSLSPGERATLSCRASQSVSSSYLVWYQQKPGQAPRLLIYGASSRAPGIPDRFSGSGSGTDFTLTISRLEREDFAVYYCQQYGRSFGQGTKVEIK; and combination thereof.

In some embodiments, the plurality of neutralizing antibodies against HA head comprise antibodies selected from the group consisting of:
antibody comprising a heavy chain having an amino acid sequence of EVQLVQSGAEVKKPGASVKVSCKASGYTFTDYHINWVRQAPGQGLEWMGWIHPNSGDTNYAQKFQGWVTMTRDTAISTAYMEVNGLKSDDTAVYYCARGGLEPRSVDYYYYGMDVWGQGTTVTVSS; and a light chain having an amino acid sequence of QSVLTQPPSVSVAPGQTARITCGGNDIGRKSVHWNQQKPGQAPVLVVCYDSDRPSGIPERFSGSNSGNTATLTISRVEAGDEADYYCQVWDSSSDHVIFGGGTKLTVL;
antibody comprising a heavy chain having an amino acid sequence of EVQLVESGPGLVKPSDILSLTCAVSGYSISSNYYWGWIRQPPGKGLEWIGSIYHSGSTYYKPSLESRLGISVDTSKNQFSLKLSFVSAADTAVYYCARHVRSGYPDTAYYFDKWGQGTLVTVSs; and a light chain having an amino acid sequence of TSYVLTQPPSVSVAPGETARISCGGNNIGTKVLHWYQQTPGQAPVLVVYDDSDRPSGIPERFSGSNSGNTATLTISRVEVGDEADYYCQVWDISTDQAVFGGGTKLTVL;
antibody comprising a heavy chain having an amino acid sequence of EVQLVESGGGLVQPGGSLRLSCAASEFNFKSYWMTWVRQAPGKGLEWVANINQDGSEKNYVDSVKGRFTISRDNAKNSLHLQMSSLRVDDTAVYYCARTGSSWDTYYYYYAMDVWGQGTTVTVSS; and a light chain having an amino acid sequence of DIQLTQSPVSLSLSPGERATLSCRASQSVSSSYLVWYQQKPGQAPRLLIYGASSRAPGIPDRFSGSGSGTDFTLTISRLEREDFAVYYCQQYGRSFGQGTKVEIK; and combination thereof.

In some embodiments, the plurality of neutralizing antibodies against HA head comprise antibodies that compete with one or more antibodies described herein (e.g., monoclonal antibodies CH65, 5J8, and/or 4K8).

In some embodiments, individual neutralizing antibodies or secondary antibodies recognizing the individual neutralizing antibodies are labeled with a detectable entity. In some embodiments, the bindings between individual neutralizing antibodies and the engineered HA antigen are detected by detecting detectable signal generated by the detectable entity. In some embodiments, the detectable signal is a fluorescent signal. In some embodiments, the binding levels between individual neutralizing antibodies and the engineered HA antigen displayed on the surface of the one or more cells are detected by flow cytometry. In some embodiments, the binding levels are quantitatively detected.

In some embodiments, the methods of the present invention further comprise a step of down-selecting the engineered HA antigen as properly expressed if the binding levels are 50% or greater compared to a wild-type benchmark for at least three neutralizing antibodies against HA stem. In some embodiments, the wild-type benchmark is defined by the binding levels between the individual neutralizing antibodies and a wild-type HA used for engineering the engineered HA.

In some embodiments, the method further comprises a step of down-selecting the engineered HA antigen as properly folded if the binding levels are over background for at least one neutralizing antibody against HA head and at least three neutralizing antibodies against HA stem. In some embodiments, the engineered HA antigen is down-selected as properly folded if the binding levels are at least 2, 3, 4, or 5 times higher over background for at least one neutralizing antibody against HA head and at least three neutralizing antibodies against HA stem. In some embodiments, the background is defined by a cell that does not contain the nucleic acid sequence encoding the engineered HA antigen. In some embodiments, the method further comprises a step of predicting specificity of the down selected engineered HA antigen against influenza strain clusters based on the binding levels by the neutralizing antibodies against HA head. In some embodiments, the method further comprises a step of testing immunogenicity of the down-selected engineered HA antigen.

In another aspect, the present invention provides an engineered hemagglutinin (HA) antigen down-selected by a method described herein. In still another aspect, the present invention further provides an influenza vaccine comprising an engineered hemagglutinin (HA) antigen down-selected by a method described herein. In some embodiments, the influenza vaccine comprises a viral-like particle. In some embodiments, the influenza vaccine is a live attenuated virus. In some embodiments, the influenza vaccine is a recombinant protein. In some embodiments, the influenza vaccine is capable of eliciting broadly cross-neutralizing antibodies.

In yet another aspect, the present invention provides kits for analyzing expression and conformation of engineered hemagglutinin (HA) antigens using various methods described herein. In some embodiments, a kit of the invention includes a panel of neutralizing antibodies comprising a plurality of neutralizing antibodies against HA stem and a plurality of neutralizing antibodies against HA head as described herein.

Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### BRIEF DESCRIPTION OF THE DRAWING

The Drawing included herein, which is comprised of the following Figures, is for illustration purposes only not for limitation.

FIG. 1 depicts an exemplary scheme comparing the steps of traditional HA screening (top) with that of rapid screening of HA antigen expression and conformation as embodied by the present invention (bottom).

FIG. 2 depicts an exemplary scheme illustrating the steps for screening an engineered HA antigen including transfection, expression, and characterization with broadly neutralizing antibodies and flow cytometry.

FIG. 3 depicts a tertiary structure of an HA antigen.

FIG. 4A depicts exemplary data showing antibody-binding levels as compared to HA benchmark.

FIG. 4B depicts exemplary down-selection results based on expression and conformation criteria.

FIG. 5 depicts exemplary immunogenicity analysis data of two down-selected HA antigens SP-007 and SP-009.

FIG. 6 depicts exemplary data illustrating the expression and conformation screening results over 200 engineered HA antigens designed using 4 different engineering methods.

### DEFINITIONS

In order for the present invention to be more readily understood, certain terms are first defined below. Additional definitions for the following terms and other terms are set forth through the specification.

Adjuvant: As used herein, the term “adjuvant” refers to a substance or vehicle that non-specifically enhances the immune response to an antigen. Adjuvants can include a suspension of minerals (alum, aluminum hydroxide, or phosphate) on which antigen is adsorbed; or water -in-oil emulsion in which antigen solution is emulsified in mineral oil (for example, Freund's incomplete adjuvant), sometimes with the inclusion of killed mycobacteria (Freund's complete adjuvant) to further enhance antigenicity. Immunostimulatory oligonucleotides (such as those including a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants also include biological molecules, such as costimulatory molecules. Exemplary biological adjuvants include IL-2, RANTES, GM-CSF, TNF-a, IFN-y, G-CSF, LFA-3, CD72, B7-1, B7-2, OX-40L and 41 BBL.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Antibody: As used herein, the term “antibody” refers to a polypeptide that includes canonical immunoglobulin sequence elements sufficient to confer specific binding to a particular target antigen. In some embodiments, as used herein, the term “antibody” also refers to an “antibody fragment” or “antibody fragments”, which includes a portion of an intact antibody, such as, for example, the antigen-binding or variable region of an antibody. Examples of “antibody fragments” include Fab, Fab′, F(ab′)2, and Fv fragments; triabodies; tetrabodies; linear antibodies; single-chain antibody molecules; and CDR-containing moieties included in multi-specific antibodies formed from antibody fragments. Those skilled in the art will appreciate that the term “antibody fragment” does not imply and is not restricted to any particular mode of generation. An antibody fragment may be produced through use of any appropriate methodology, including but not limited to cleavage of an intact antibody, chemical synthesis, recombinant production, etc. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (V_{II}) domain (located at the tips of the Y structure), followed by three constant domains: C_{H}1, C_{H}2, and the carboxy-terminal C_{H}3 (located at the base of the Y's stem). A short region, known as the “switch”, connects the heavy chain variable and constant regions. The “hinge” connects C_{H}2 and C_{H}3 domains to the rest of the antibody. Two disulfide bonds in this hinge region connect the two heavy chain polypeptides to one another in an intact antibody. Each light chain is comprised of two domains—an amino-terminal variable (V_{L}) domain, followed by a carboxy-terminal constant (C_{L}) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the C_{H}2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. Amino acid sequence comparisons among antibody polypeptide chains have defined two light chain (κ andλ ) classes, several heavy chain (e.g., μ, λ, α, ε, δ ) classes, and certain heavy chain subclasses (α1, α2, γ1γ2, γ3, and γ4). Antibody classes (IgA [including IgA1, IgA2], IgD, IgE, IgG [including IgG1, IgG2, IgG3, IgG4], IgM) are defined based on the class of the utilized heavy chain sequences. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is monoclonal; in some embodiments, an antibody is polyclonal. In some embodiments, an antibody has constant region sequences that are characteristic of mouse, rabbit, primate, or human antibodies. In some embodiments, an antibody sequence elements are humanized, primatized, chimeric, etc., as is known in the art. Moreover, the term “antibody” as used herein, will be understood to encompass (unless otherwise stated or clear from context) can refer in appropriate embodiments to any of the art-known or developed constructs or formats for capturing antibody structural and functional features in alternative presentation. For example, in some embodiments, the term can refer to bi- or other multi-specific (e.g., zybodies, etc.) antibodies, Small Modular ImmunoPharmaceuticals (“SMIPs™ ”), single chain antibodies, camelid antibodies, and/or antibody fragments. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc.], or other pendant group [e.g., poly-ethylene glycol, etc.]).

Antigen: As used herein, the term “antigen”, refers to an agent that elicits an immune response; and/or (ii) an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism; alternatively or additionally, in some embodiments, an antigen elicits a cellular response (e.g., involving T-cells whose receptors specifically interact with the antigen) in an organism. It will be appreciated by those skilled in the art that a particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the members of a target organism species. In some embodiments, an antigen binds to an antibody and/or T cell receptor, and may or may not induce a particular physiological response in an organism. In some embodiments, for example, an antigen may bind to an antibody and/or to a T cell receptor in vitro, whether or not such an interaction occurs in vivo. In some embodiments, an antigen reacts with the products of specific humoral or cellular immunity, including those induced by heterologous immunogens. In some embodiments of the disclosed compositions and methods, an influenza HA protein is an antigen.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility of the disease, disorder, or condition (e.g., across a relevant population). In some embodiments, two or more entities are physically “associated” with one another if they interact, directly or indirectly, so that they are and remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof. Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can be assessed in any of a variety of contexts—including where interacting entities or moieties are studied in isolation or in the context of more complex systems (e.g., while covalently or otherwise associated with a carrier entity and/or in a biological system or cell).

Biological activity: As used herein, the phrase “biological activity” refers to an observable biological effect or result achieved by an agent or entity of interest. For example, in some embodiments, a specific binding interaction is a biological activity. In some embodiments, modulation (e.g., induction, enhancement, or inhibition) of a biological pathway or event is a biological activity. In some embodiments, presence or extent of a biological activity is assessed through detection of a direct or indirect product produced by a biological pathway or event of interest.

Carrier: As used herein, the term “carrier” refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Characteristic Portion: As used herein, the term “characteristic portion” is used, in the broadest sense, to refer to a portion of a substance whose presence (or absence) correlates with presence (or absence) of a particular feature, attribute, or activity of the substance. In some embodiments, a characteristic portion of a substance is a portion that is found in the substance and in related substances that share the particular feature, attribute or activity, but not in those that do not share the particular feature, attribute or activity.

Characteristic Pandemic Feature: As used herein the term “characteristic pandemic feature” is one that is found in at least one reference pandemic strain and not in at least one non-pandemic strain. In some embodiments, a characteristic pandemic feature is one that is commonly found in pandemic strains and rarely found in non-pandemic strains.

Characteristic sequence element: As used herein, the phrase “characteristic sequence element” refers to a sequence element found in a polymer (e.g., in a polypeptide or nucleic acid) that represents a characteristic portion of that polymer. In some embodiments, presence of a characteristic sequence element correlates with presence or level of a particular activity or property of the polymer. In some embodiments, presence (or absence) of a characteristic sequence element defines a particular polymer as a member (or not a member) of a particular family or group of such polymers. A characteristic sequence element typically comprises at least two monomers (e.g., amino acids or nucleotides). In some embodiments, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some embodiments, a characteristic sequence element includes at least first and second stretches of continuous monomers spaced apart by one or more spacer regions whose length may or may not vary across polymers that share the sequence element.

Corresponding to: As used herein, the term “corresponding to” is often used to designate the position/identity of an amino acid residue in a polypeptide of interest (e.g., an HA polypeptide). Those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190th amino acid in a particular amino acid chain but rather corresponds to the residue found at 190 in the reference polypeptide; those of ordinary skill in the art readily appreciate how to identify “corresponding” amino acids. Typically, residues in HA polypeptides are designated with reference to a canonical wild type H1 HA, and reference in a polypeptide of interest that correspond to resides in the canonical wild type H1 HA are described using the numbering of the residues to which they correspond.

Determine: Many methodologies described herein include a step of “determining”. Those of ordinary skill in the art, reading the present specification, will appreciate that such “determining” can utilize any of a variety of techniques available to those skilled in the art, including for example specific techniques explicitly referred to herein. In some embodiments, a determination involves manipulation of a physical sample. In some embodiments, a determination involves consideration and/or manipulation of data or information, for example utilizing a computer or other processing unit adapted to perform a relevant analysis. In some embodiments, a determination involves receiving relevant information and/or materials from a source. In some embodiments, determining involves comparing one or more features of a sample or entity to a comparable reference.

Direct-binding amino acids: As used herein, the phrase “direct-binding amino acids” refers to HA polypeptide amino acids which interact directly with one or more glycans that is/are associated with host cell HA receptors.

Eliciting broadly cross-neutralizing antibodies: The phrase “eliciting broadly cross-neutralizing antibodies”, as used herein, refers to the ability of an influenza antigen to cause an adaptive immune response resulting in the production of a plurality of antibodies that are capable of neutralizing (e.g., blocking infectivity) wild-type HA antigens from a variety of influenza types (e.g. influeanza A or B), subtypes (e.g., influenza A H1N1, H3N2, H5N1, etc.), and/or strains (e.g., A/California/07/2009, A/USSR/90/1977, A/Brazil/11/1978, A/Chile/1/1983, A/Taiwan/1/1986 A/Beijing/262/1995, A/New Caledonia/20/1999, A/Solomon Island/3/2006, and A/Brisbane/59/2007). The breadth of neutralization may be determined by testing the ability of antibodies to neutralize hemagglutination activity and/or infectivity of a panel of influenza strains, each strain expressing a different wild-type HA sequence.

Engineered: The term “engineered”, as used herein, describes a polypeptide whose amino acid sequence has been designed by man and/or whose existence and production require action of the hand of man. For example, an engineered HA polypeptide has an amino acid sequence that differs from the amino acid sequences of HA polypeptides found in natural influenza isolates. In some embodiments, an engineered HA polypeptide has an amino acid sequence that differs from the amino acid sequence of HA polypeptides included in the NCBl database.

Epitope: As used herein, the term “epitope” includes any moiety that is specifically recognized by an immunoglobulin (e.g., antibody or receptor) binding component in whole or in part. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Excipient: As used herein, the term “excipient” refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Expression: The term “expression”, when used in reference to a nucleic acid herein, refers to one or more of the following events: (1) production of an RNA transcript of a DNA template (e.g., by transcription); (2) processing of an RNA transcript (e.g., by splicing, editing, 5′ cap formation, and/or 3′ end formation); (3) translation of an RNA into a polypeptide; and/or (4) post-translational modification of a polypeptide.

Hemagglutinin (HA) polypeptide: As used herein, the term “hemagglutinin polypeptide” (or “HA polypeptide') refers to a polypeptide whose amino acid sequence includes at least one characteristic sequence of HA. A wide variety of HA sequences from influenza isolates are known in the art; indeed, the National Center for Biotechnology Information (NCBI) maintains a database (http://www.ncbi.nlm.nih.gov/genomes/FLU/) that, as of the filing of the present application included at least 9796 HA sequences. Those of ordinary skill in the art, referring to this database, can readily identify sequences that are characteristic of HA polypeptides generally, and/or of particular HA polypeptides (e.g., H1, H2, H3, H4, H5, H6, H7, H8, H9, H10, H11, H12, H13, H14, H15, or H16 polypeptides; or of HAs that mediate infection of particular hosts, e.g., avian, camel, canine, cat, civet, environment, equine, human, leopard, mink, mouse, seal, stone martin, swine, tiger, whale, etc.). For example, in some embodiments, an HA polypeptide includes one or more characteristic sequence elements found between about residues 97 and about 185, about 324 and about 340, about 96 and about 100, and/or about 130 and about 230 of an HA protein found in a natural isolate of an influenza virus.

H1N1HA polypeptide: An “H1N1 HA polypeptide”, as that term is used herein, is an HA polypeptide whose amino acid sequence includes at least one sequence element that is characteristic of H1N1 and distinguishes H1N1 from other HA subtypes. Representative such sequence elements can be determined by alignments as will be understood by those skilled in the art.

Host: The term “host” is used herein to refer to a system (e.g., a cell, organism, etc) in which a polypeptide of interest is present. In some embodiments, a host is a system that is susceptible to infection with a particular infectious agent. In some embodiments, a host is a system that expresses a particular polypeptide of interest.

Host cell: As used herein, the phrase “host cell” refers to a cell into which exogenous DNA (recombinant or otherwise) has been introduced. For example, host cells may be used to produce the engineered influenza hemagglutinin polypeptides described herein by standard recombinant techniques. Persons of skill upon reading this disclosure will understand that such terms refer not only to the particular subject cell, but, to the progeny of such a cell. Because certain modifications may occur in succeeding generations due to either mutation or environmental influences, such progeny may not, in fact, be identical to the parent cell, but are still included within the scope of the term “host cell” as used herein. In some embodiments, host cells include any prokaryotic and eukaryotic cells that are suitable for expressing an exogenous DNA (e.g., a recombinant nucleic acid sequence). Exemplary cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, the cell is a human, monkey, ape, hamster, rat, or mouse cell. In some embodiments, the cell is eukaryotic and is selected from the following cells: CHO (e.g., CHO K1, DXB-11 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3A cell, HT1080 cell, myeloma cell, tumor cell, and a cell line derived from an aforementioned cell. In some embodiments, the cell comprises one or more viral genes, e.g., a retinal cell that expresses a viral gene (e.g., a PER.C6™ cell).

Immune response: As used herein, the term “immune response” refers to a response of a cell of the immune system, such as a B cell, T cell, dendritic cell, macrophage or polymorphonucleocyte, to a stimulus such as an antigen or vaccine. An immune response can include any cell of the body involved in a host defense response, including for example, an epithelial cell that secretes an interferon or a cytokine An immune response includes, but is not limited to, an innate and/or adaptive immune response. As used herein, a protective immune response refers to an immune response that protects a subject from infection (prevents infection or prevents the development of disease associated with infection). Methods of measuring immune responses are well known in the art and include, for example, measuring proliferation and/or activity of lymphocytes (such as B or T cells), secretion of cytokines or chemokines, inflammation, antibody production and the like.

Immunogen: As used herein, the term “immunogen” refers to a compound, composition, or substance which is capable, under appropriate conditions, of stimulating an immune response, such as the production of antibodies or a T cell response in an animal, including compositions that are injected or absorbed into an animal. As used herein, an “immunogenic composition” is a composition comprising an immunogen (such as an HA polypeptide). As used herein, “immunize” means to render a subject protected from an infectious disease, such as by vaccination.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Influenza virus: As used herein, the term “influenza virus” refers to a segmented negative-strand RNA virus that belongs to the Orthomyxoviridae family. There are three types of Influenza viruses, A, B, and C. Influenza A viruses infect a wide variety of birds and mammals, including humans, horses, marine mammals, pigs, ferrets, and chickens. In animals, most influenza A viruses cause mild localized infections of the respiratory and intestinal tract. However, highly pathogenic influenza A strains, such as H5N1, cause systemic infections in poultry in which mortality may reach 100%. In 2009, H1N1 influenza was the most common cause of human influenza. A new strain of swine origin H1N1 emerged in 2009 and was declared pandemic by the World Health Organization. This strain was referred to as “swine flu.” H1N1 influenza A viruses were also responsible for the Spanish flu pandemic in 1918, the Fort Dix outbreak in 1976, and the Russian flu epidemic in 1977-1978.

Influenza vaccine: As used herein, the term “influenza vaccine” refers to an immunogenic composition capable of stimulating an immune response, administered for the prevention, amelioration, or treatment of influenza virus infection. An influenza vaccine may include, for example, attenuated or killed influenza virus, virus-like particles (VLPs) and/or antigenic polypeptides (e.g., the engineered hemagglutinins described herein) or DNA derived from them, or any recombinant versions of such immunogenic materials.

Isolated: As used herein, the term “isolated” refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide.

Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

Outbreak: As used herein, an influenza virus “outbreak” refers to a collection of virus isolates from within a single country in a given year.

Pandemic strain: A “pandemic” influenza strain is one that has caused or has capacity to cause pandemic infection of human populations. In some embodiments, a pandemic strain has caused pandemic infection. In some embodiments, such pandemic infection involves epidemic infection across multiple territories; in some embodiments, pandemic infection involves infection across territories that are separated from one another (e.g., by mountains, bodies of water, as part of distinct continents, etc) such that infections ordinarily do not pass between them.

Prevention: The term “prevention”, as used herein, refers to prophylaxis, avoidance of disease manifestation, a delay of onset, and/or reduction in frequency and/or severity of one or more symptoms of a particular disease, disorder or condition (e.g., infection for example with influenza virus). In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition.

Receptor-Binding Site (RBS): As used herein, the term “receptor-binding site” or “RBS” comprises contiguous or non-contiguous amino acid residues of the head region of an influenza HA polypeptide, which include amino acids involved in direct binding of sialic acid on the target cell receptor proteins. Amino acid residues that make up a “receptor-binding site” or “RBS” of an influenza HA polypeptide may be described from crystal structures of HA polypeptides complexed with sialic acid analogs and identifying amino acid residues within a certain proximity to the analog or may be described in reference to an HA polypeptide sequence from a particular viral strain (e.g., A/New Caledonia/20/99 or A/California/07/2009). Thus, in some embodiments, the “receptor-binding site” or “RBS” of an engineered HA polypeptide as described herein may be determined using a reference HA polypeptide sequence. In some embodiments, the “receptor-binding site” or “RBS” of an engineered HA polypeptide as described herein may be determined using the crystal structures of HA polypeptide sequence in complex with human and avian receptor analogs (ex. LSTa, LSTc). An exemplary reference crystal structure of HA polypeptide sequence in complex with LSTc includes A/Puerto Rico/8/1934 (H1N1) pdb|1RVZ.

Recombinant: As used herein, the term “recombinant” is intended to refer to polypeptides (e.g., HA polypeptides as described herein) that are designed, engineered, prepared, expressed, created or isolated by recombinant means, such as polypeptides expressed using a recombinant expression vector transfected into a host cell, polypeptides isolated from a recombinant, combinatorial polypeptide library or polypeptides prepared, expressed, created or isolated by any other means that involves splicing selected sequence elements to one another. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source. In some embodiments, one or more such selected sequence elements results from the combination of multiple (e.g., two or more) known sequence elements that are not naturally present in the same polypeptide (e.g., two epitopes from two separate HA polypeptides).

Recombinant influenza vaccine: As used herein, the term “recombinant influenza vaccine” refers to influenza-specific immunogenic composition comprising the engineered influenza hemagglutinins described herein, including but not limited to, influenza virus, subunit preparations thereof, virus-like particles, recombinant protein (i.e., preparations composed of recombinant HA purified to varying degree), and DNA-and viral vector-based vaccines. Recombinant influenza vaccines as described herein may optionally contain one or more adjuvants.

Specificity: As is known in the art, “specificity” is a measure of the ability of a particular ligand (e.g., an antibody, an HA polypeptide, etc) to distinguish its binding partner (e.g., an antigen, a human HA receptor, and particularly a human upper respiratory tract HA receptor) from other potential binding partners (e.g., an avian HA receptor).

Subject: As used herein, the term “subject” means any mammal, including humans. In certain embodiments of the present invention the subject is an adult, an adolescent or an infant. In some embodiments, terms “individual” or “patient” are used and are intended to be interchangeable with “subject”. Also contemplated by the present invention are the administration of the pharmaceutical compositions and/or performance of the methods of treatment in-utero.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially Similar: As used herein, the term “substantially similar” refers to a comparison between two entities. In general, entities are considered to be “substantially similar” to one another when they share sufficient structural similarity (e.g., a characteristic structural feature) that they have a comparable likelihood of sharing one or more additional attributes or features. To give but one example, a characteristic, for example, glycosylation site pattern, being either the same or similar enough between two influenza strains, that the human pandemic risk of each strain is the same.

Substantial sequence homology: The phrase “substantial homology” is used herein to refer to a comparison between amino acid or nucleic acid sequences. As will be appreciated by those of ordinary skill in the art, two sequences are generally considered to be “substantially homologous” if they contain homologous residues in corresponding positions. Homologous residues may be identical residues. Alternatively, homologous residues may be non-identical residues will appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains. Substitution of one amino acid for another of the same type may often be considered a “homologous” substitution. Typical amino acid categorizations are summarized in Table 1 and 2.

TABLE 1

| | | | | | |
|---|---|---|---|---|---|
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | nonpolar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

TABLE 2

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul, et al., “Gapped BLAST and PSI-BLAST: a new generation of protein database search programs”, Nucleic Acids Res. 25:3389-3402, 1997; Baxevanis, et al., Bioinformatics : A Practical Guide to the Analysis of Genes and Proteins, Wiley, 1998; and Misener, et al., (eds.), Bioinformatics Methods and Protocols (Methods in Molecular Biology, Vol. 132), Humana Press, 1999; all of the foregoing of which are incorporated herein by reference. In addition to identifying homologous sequences, the programs mentioned above typically provide an indication of the degree of homology. In some embodiments, two sequences are considered to be substantially homologous if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are homologous over a relevant stretch of residues. In some embodiments, the relevant stretch is a complete sequence. In some embodiments, the relevant stretch is at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500 or more residues.

Substantial identity: The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul et al., Nucleic Acids Res. 25:3389-3402, 1997; Baxevanis et al., Bioinformatics : A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues. In the context of an HA polypeptide, reference to “substantial identity” typically refers to a HA polypeptide (or HA epitope) having an amino acid sequence at least 90%, preferably at least 91%, at least 92%, at least 93%, at least 94%, at least 95%, at least 96%, at least 97%, at least 98%, or at least 99% identical to that of a reference HA polypeptide (or HA epitope).

Vaccination: As used herein, the term “vaccination” refers to the administration of a composition intended to generate an immune response, for example to a disease-causing agent. Vaccination can be administered before, during, and/or after exposure to a disease-causing agent, and/or to the development of one or more symptoms, and in some embodiments, before, during, and/or shortly after exposure to the agent. In some embodiments, vaccination includes multiple administrations, appropriately spaced in time, of a vaccinating composition.

Variant: As used herein, the term “variant” is a relative term that describes the relationship between a particular polypeptide of interest and a “parent” or “reference” polypeptide to which its sequence is being compared. A polypeptide of interest is considered to be a “variant” of a parent or reference polypeptide if the polypeptide of interest has an amino acid sequence that is identical to that of the parent but for a small number of sequence alterations at particular positions. Typically, fewer than 20%, 15%, 10%, 9%, 8%, 7%, 6%, 5%, 4%, 3%, 2% of the residues in the variant are substituted as compared with the parent. In some embodiments, a variant has 10, 9, 8, 7, 6, 5, 4, 3, 2, or 1 substituted residue as compared with a parent. Often, a variant has a very small number (e.g., fewer than 5, 4, 3, 2, or 1) number of substituted functional residues (i.e., residues that participate in a particular biological activity). Furthermore, a variant typically has not more than 5, 4, 3, 2, or 1 additions or deletions, and often has no additions or deletions, as compared with the parent. Moreover, any additions or deletions are typically fewer than about 25, about 20, about 19, about 18, about 17, about 16, about 15, about 14, about 13, about 10, about 9, about 8, about 7, about 6, and commonly are fewer than about 5, about 4, about 3, or about 2 residues. In some embodiments, the parent or reference polypeptide is one found in nature. As will be understood by those of ordinary skill in the art, a plurality of variants of a particular polypeptide of interest may commonly be found in nature, particularly when the polypeptide of interest is an infectious agent polypeptide.

Virus-like paricle (VLP): As used herein, the phrase “virus-like particle” or “VLP” refers to particles that resemble a virus yet lack any viral genetic material and, therefore, are not infectious. A “virus-like particle” or “VLP” may be produced by heterologous expression in a variety of cell culture systems including mammalian cell lines, insect cell lines, yeast, and plant cells. In addition, VLPs can be purified by methods known in the art. In some embodiments, an influenza VLP as described herein comprises hemagglutinin (HA) polypeptides and neuraminidase (NA) polypeptides. In some embodiments, an influenza VLP as described herein comprises HA polypeptides, NA polypeptides and/or viral structural polypeptides (e.g., an influenza structural protein such as influenza Ml). In some certain embodiments, an influenza VLP as described herein comprises HA polypeptides, NA polypeptides and/or M1 polypeptides. In some embodiments, an influenza VLP as described herein comprises HA polypeptides, NA polypeptides and/or HIVgag polypeptides. As persons of skill are aware, other viral structural proteins may be used as alternatives to those exemplified herein. Influenza VLPs can be produced by transfection of host cells (e.g., mammalian cells) with plasmids encoding HA and NA proteins, and optionally HIVgag proteins. After incubation of the transfected cells for an appropriate time to allow for protein expression (such as for approximately 72 hours), VLPs can be isolated from cell culture supernatants. In some embodiments, influenza VLPs as described herein are produced by transient transfection in mammalian cells (e.g., human cells). In some embodiments, influenza VLPs are analyzed by the use of one or more assays. To give but a few examples, influenza VLPs may be analyzed for hemagglutinin activity, dynamic light scattering and hemmagglutinin content quantitation by protein staining Other assays will be readily apparent to persons of skill upon reviewing the present disclosure.

Wild type: As is understood in the art, the phrase “wild type” generally refers to a normal form of a protein or nucleic acid, as is found in nature. For example, wild type HA polypeptides are found in natural isolates of influenza virus. A variety of different wild type HA sequences can be found in the NCBI influenza virus sequence database, available through the World Wide Web at ncbi.nlm.nih.gov/genomes/FLU/FLU.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference for any purpose.

### DETAILED DESCRIPTION OF CERTAIN EMBODIMENTS

The present invention provides, among other things, methods of analyzing expression and/or conformation of engineered hemagglutinin (HA) antigens for use in universal influenza vaccines. In some embodiments, inventive methods described herein are based on immunostaining of cells with a panel of neutralizing antibodies under conditions that permit the neutralizing antibodies to bind to engineered HA antigens displayed on surface of the cells. In some embodiments, the binding levels between the neutralizing antibodies and the engineered HA antigens may be detected by flow cytometry and the detected binding levels may be used to determine if the engineered HA antigens are properly expressed and/or folded. In some embodiments, a panel of neutralizing antibodies suitable for the present invention includes a plurality of neutralizing antibodies against HA stem and a plurality of neutralizing antibodies against HA head.

Various aspects of the invention are described in further detail in the following subsections. The use of subsections is not meant to limit the invention. Each subsection may apply to any aspect of the invention. In this application, the use of “or” means “and/or” unless stated otherwise.

### Engineered Hemagglutinin (HA) Antigens

The present invention may be used to screen, analyze, identify, select and/or validate HA antigens engineered by any methods including, but not limited to, in silico designed HA antigens.

Hemagglutinin (HA) is a glycoprotein responsible for the binding of the influenza virus to cells with sialic acid on the membranes, and is highly variable across influenza virus strains. Indeed, currently marketed influenza vaccines typically need to be updated annually based on predicted strains that will be present in human populations in the impending season. To provide more effective vaccination against influenza, universal vaccines with increased breadth of immunity have been developed based on conserved and/or cross-reactive epitopes using bioinformatics tools. To give but a few examples, engineered HA antigens may be produced using methodology such as computationally optimized broadly reactive antigens (e.g., COBRA; see WO2012/177760, herein incorporated by reference), engineered mosaic hemagglutinin polypeptides (see U.S. Provisional Patent Application entitled “Engineered Influenza Hemagglutinin Polypeptides and Immunogenic Compositiosn Thereof” filed on even date, herein incorporated by reference), reverse genetics using plasmids encoding influenza sense RNA and/or mRNA (see Subbarao and Katz, 2004, Curr. Top. Microbiol. 283:313-342), protein engineering, influenza consensus sequences based on various influenza strains (e.g. currently circulating, pandemic, pre-pandemic, etc.) or combinations of influenza strains, deletion and/or rearrangement of structural domains, swapping of structural domains derived from various strains, combinations of neutralizing hemagglutinin B-cell epitope patterns derived from multiple influenza strains, combination of various cross-reactive epitopes among multiple influenza strains, etc. Upon reading the present disclosure, persons of skill will understand that inventive methods described herein may be employed to screen, analyze, identify, select and/or validate any HA antigen designed by any method known in the art or described herein.

Engineered HA antigens generated from computational-based approaches may be based on consensus sequences generated by alignment of various influenza strains. Such influenza strains may be grouped according to various criteria such as, for example, a year (or range) of isolation (e.g., 1918, 1945-1950, etc.), specific sequence features (e.g., glycosylation sites), species specificity (e.g., avian, swine, etc.), and/or combinations thereof. Further refinement of candidate HA sequence may be performed based multiple consensus sequences (e.g., primary, secondary, tertiary, etc.) to arrive a final engineered HA sequence. In structure-based approaches, engineered HA polypeptides can be generated from multiple epitopes from multiple viral isolates as possible. Such designs for engineered HA polypeptides are based on combinations of multiple B cell epitopes from different hemagglutinin sequences (e.g., influenza A; influenza B; influenza A subtypes H1, H2, H3, H5, H7, etc.; or of HAs that mediate infection of particular hosts, e.g., avian, camel, canine, cat, civet, environment, equine, human, leopard, mink, mouse, seal, stone martin, swine, tiger, whale, etc.) into mosaic antigens. Multiple approaches can be employed to deduce epitope patterns that are combined to generate an engineered mosaic HA antigens.

Various candidate HA antigens may be generated in host cells using the above criteria and subsequently expressed in cells (e.g., mammalian cells) for further testing and development. Once the candidate HA antigens (or VLPs, or vaccines) have been engineered using a computational approach and expressed in a host cell, they may be analyzed using the methods described herein for determining their conformation and suitability for use in eliciting an immune response in a subject. Subsequent animal studies may be performed to assess immunogenicity of engineered HA antigens developed using computational-based approaches.

### Cells Expressing Engineered HA Antigens

According to the present invention, cells expressing engineered HA antigens may be used in a screening assay. In some embodiments, the amino acid sequences of engineered influenza HA antigens may be back-translated, optimized for protein expression and resulting nucleic acid molecules are inserted into a vector (e.g., a plasmid) that is able to express the HA antigens when introduced into an appropriate host cell.

Any of the methods known to one skilled in the art for the insertion of DNA fragments into a vector may be used to construct expression vectors encoding the fusion proteins of the present invention under control of transcriptional/translational control signals. These methods may include in vitro recombinant DNA and synthetic techniques and in vivo recombination (See Sambrook et al. Molecular Cloning, A Laboratory Manual, Cold Spring Harbor Laboratory; Current Protocols in Molecular Biology, Eds. Ausubel, et al., Greene Publ. Assoc., Wiley-Interscience, NY).

In some embodiments, nucleic acids can be DNA or RNA, and can be single stranded or double-stranded. In some embodiments, nucleic acids in accordance with the invention may include one or more non-natural nucleotides; in some embodiments, nucleic acids in accordance with the invention include only natural nucleotides.

Expression of nucleic acid molecules in accordance with the present invention may be regulated by a second nucleic acid sequence so that the molecule is expressed in a host transformed with the recombinant DNA molecule. For example, expression of the nucleic acid molecules of the invention may be controlled by a promoter and/or enhancer elements, which are known in the art.

Nucleic acid constructs of the present invention are inserted into an expression vector such as a plasmid or viral vector by methods known to the art, and nucleic acid molecules are operatively linked to an expression control sequence.

An expression vector containing a nucleic acid molecule may be transformed or transfected into a suitable host cell to allow for expression of the engineered HA protein encoded by the nucleic acid constructs on the surface of the cell. Various cell types may be used including, but not limited to, mammalian cells, insect cells, yeast cells, microalgae, plant cells and bacterial cells. Insect cells useful for producing influenza vaccines include, but are not limited to: SF cells, caterpillar cells, butterfly cells, moth cells, SF9 cells, SF21 cells, drosophila cells, S2 cells, fall armyworm cells, cabbage looper cells, Spodoptera frugiperda cells, and Trichoplasia ni cells. Suitable mammalian cells for producing influenza vaccines include, but are not limited to: Madin-Darby canine kidney (MDCK) cells, VERO cells, EBx cells, chicken embryo cells, Chinese hamster ovary (CHO) cells, monkey kidney cells, human embryonic kidney cells, HEK293T cells, NS0 cells, myeloma cells, hybridoma cells, primary adenoid cell lines, primary bronchial epithelium cells, transformed human cell lines, and Per.C6 cells. Other useful systems include microalgae (e.g. Schizochytrium sp.; see, e.g., Bayne, A-C.V. et al., PLOS ONE, 8(4):e61790, April 2013), plant-based systems (e.g., tobacco plants; see, e.g., Jul-Larsen, A., et al., Hum Vaccin Immunother., 8(5):653-61, 2012), yeast (see, e.g., Athmaram, T.N. et al., Virol J., 8:524, 2011), and fungi (see, e.g., Allgaier, S. et al., Biologicals, 37:128-32, 2009). Bacterial based expression systems are also encompassed by the present invention (see, e.g., Davis, A.R. et al., Gene, 21:273-284, 1983).

### Panel of Neutralizing Antibodies

Cells expressing engineered HA antigens may then be stained with a panel of neutralizing antibodies against HA antigens. In some embodiments, a panel of neutralizing antibodies suitable for the present invention includes one or more broadly neutralizing antibodies against HA antigens. As used herein, the term “broadly neutralizing antibody” encompasses any antibody that can neutralize HA antigens from multiple influenza strains. For example, a broadly neutralizing antibody is capable of neutralizing at least 2, 3, 4, 5, 6, 7, 8, 9, 10, 11, 12 or more HA antigens from distinct influenza strains, including HA antigens from contemporary strains, historical strains, pandemic strains, or combination thereof. In some embodiments, a neutralizing antibody panel of the invention may include one or more broadly neutralizing antibodies against contemporary strains, historical strains, and/or pandemic strains.

The three-dimensional structure of HA from various strains and the interaction with its cellular receptor, sialic acid, has been extensively studied (Wilson, et al, “Structure of the hemagglutinin membrane glycoprotein of influenza virus at 3A.degree. resolution” Nature 289:366-378 (1981); Weis, et al, “Structure of the influenza virus hemagglutinin complexed with its receptor, sialic acid” Nature, 333:426-431 (1988); Murphy and Webster, 1990). The HA molecule is present in the virion as a trimer. Each HA monomer (HA0) exists as two chains, HA1 and HA2, linked by a single disulfide bond. Infected host cells produce a precursor glycosylated polypeptide (HAO) with a molecular weight of about 85,000 Da, which in vivo, is subsequently cleaved into HA1 and HA2.

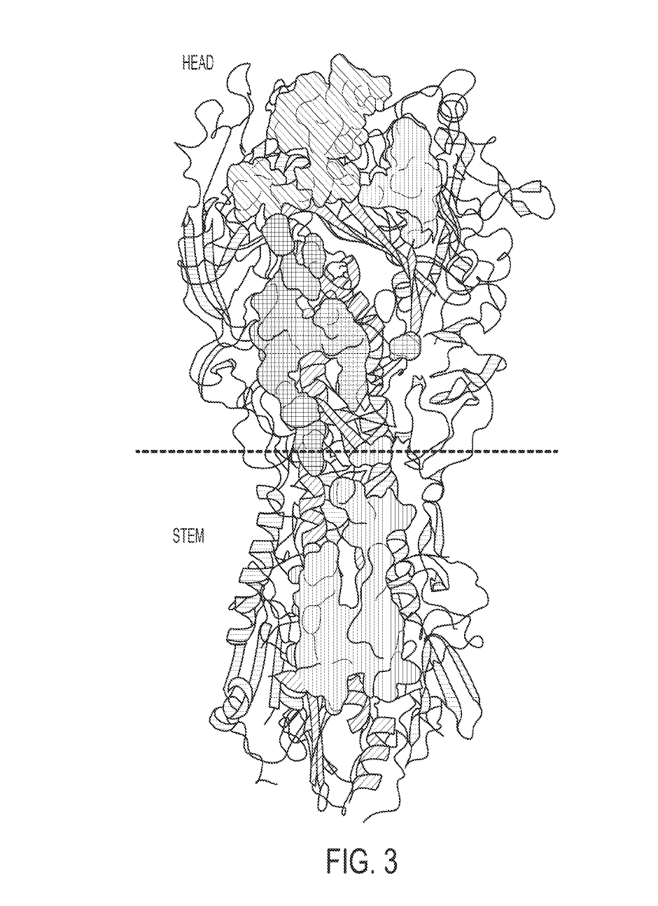

In its natural form, HA antigen is shaped like a mushroom and can be generally divided into the head and stem regions (see FIG. 3). The head region contains the receptor binding site that recognizes sialic acid receptors. It is contemplated that a panel of antibodies suitable for the present invention typically includes one or more neutralizing antibodies against HA head, in particular, those antibodies that bind to epitopes close to the receptor binding site, and one or more neutralizing antibodies against HA stem, in particular, those antibodies that bind to highly conversed conformational epitopes of the stem. In some embodiments, a suitable panel of neutralizing antibodies include at least 1, 2, 3, 4, 5, 6, 7, 8, 9, 10, 11, or 12 neutralizing antibodies against HA head. In some embodiments, a suitable panel of neutralizing antibodies include at least 1, 2, 3, 4, 5, 6, 7, 8, 9, 10, 11, or 12 neutralizing antibodies against HA stem. In some embodiments, different numbers of anti-HA head and anti-HA stem antibodies described herein may be combined to form a panel of neutralizing antibodies. For example, a suitable panel of neutralizing antibody may include at least 3 neutralizing antibodies against HA head and at least 3 neutralizing antibodies against HA stem.

Antibodies in the panel can be selected based on the attributes (position, immunogenicity, etc.) of the epitopes to which they bind. For example, antibodies can be selected to minimize redundant or overlapping epitopes in the panel, and/or to maximize immunologic relevancy. In some embodiments, the panel of antibodies against HA head can be tailored to select engineered HAs that will generate broadly neutralizing antibodies against a specific strain, for example a pandemic or seasonal influenza strain. This can be accomplished by including in the panel one or more appropriate antibodies that will bind to and select a conformationally accurate epitope for the specific strain of interest. Thus, the antibody responses generated by the engineered HAs will specifically neutralize influenza strains that match the antibody binding pattern predicted by the panel of anti-HA head broadly neutralizing antibodies.

In some embodiments, the antibody binding pattern (i.e., composition of the antibody panels) can be selected for seasonal or pandemic influenza strains. In some embodiments, the antibody binding pattern can be selected for influenza A or influenza B. In some embodiments, the antibody binding pattern can be selected for particular subtypes of influenza A, including H1N1, H1N2, H2N2, H3N1, H3N2, H5N1, H5N2, H7N7, H7N2, H7N3, H9N2 and H10N7. In some embodiments, the antibody binding pattern can be selected for particular strains of influenza A, including pandemic strains like A/New Jersey/10/1976 and A/California/07/2009, and seasonal strains such as A/Taiwan/1/1986, A/Texax/36/1991, A/Bejing/262/1995, A/New Caledonia/20/1999, A/Solomon Islands/3/2006, and A/Brisbane/59/2007.

In some embodiments, neutralizing antibodies against HA head suitable for the present invention may include antibodies that bind specifically to epitopes close to the receptor-binding site including, but not limited to, epitopes corresponding to the N-terminal end of the short a-helix, site Sa, site Sb, the edge of the receptor pocket, the C-terminus of the short a-helix and regions within 20 amino acides thereof. As a non-limiting example, an epitope close to the receptor-binding site may be defined by residues corresponding to H1N1 HA residues 133A, 137 and 222. Additional conformational epitopes close to the receptor-binding site in H1 strains are further described in Whittle et al. PNAS (2011) 108 (34): 14216-14221; Krause et al. J. Virology (2011) 86 (20): 10905-10908; Krause et al. J. Immunology (2011) 187 (7): 3704-3711; all of which are incorporated herein by reference in their entirety.

In some embodiments, neutralizing antibodies against HA stem suitable for the present invention are antibodies that bind specifically to one or more conserved epitopes in the stem region of HA from multiple influenza strains, such as, for example, conserved epitopes within A helix, which is highly conserved across all 16 subtypes. In some embodiments, such a conserved epitope may be defined by residues corresponding to HA1 residues 18, 38, 40, 42, 291-293, and 318 or a subset thereof; HA2 residues 18-21, 38, 41-43, 45, 46, 49, 52, and 56 or a subset thereof; and/or H5 residues HA2 αA, 52, 53, and 56, or a subset thereof. A helix and other conserved conformational epitopes of influenza stem are further described in Okuno et al. J. Virology (1993) 67 (5): 2552-2558; Sui et al. Nat. Stuct. & Mol. Bio. (2009) 16 (3): 265-273; and Ekiert et al. Science (2009) 324 (5924): 246-251; all of which are incorporated by reference herein in their entirety.

Antibodies known to bind to conformational epitopes (e.g., epitopes close to the receptor-binding site) of the HA head and conserved conformational epitopes (e.g., A Helix) of the HA stem may be used in an antibody panel. As non-limiting examples, suitable anti-head neutralizing antibodies may include: CH65 (contemporary strains prior 2009 pandemic) (Whittle, JRR, et al. PNAS 2011), 5J8 (contemporary and historical strains) (Krause, J C, et al. J. Virology 2011), and 4K8 (pandemic strains only) (Krause, J C, et al., J. Immunology 2011). Suitable anti-stem neutralizing antibodies may include: C179 (group 1 HAs) (Okuno, Y et al., J. Virology 1993), F10 (group 1 HAs) (Sui, et al., Nature Struct. & Mol. Bio, 2009), FI6 (group 1 and group 2 HAs) (Corti et al. Science (2011) 333 (6044): 850-856), and CR6261 (group 1 HAs) (Ekiert et al., Science, 2009). The heavy chain and light chain amino acid sequences and CDR regions of antibodies CH65, 5J8, 4K8, C179, F10, FI6, and CR6261 are described below.

CH65 Heavy Chain
EVQLVQSGAEVKKPGASVKVSCKASGYTFTDYHINWVRQAPGQGLEWMGW
IHPNSGDTNYAQKFQGWVTMTRDTAISTAYMEVNGLKSDDTAVYYCARGG
LEPRSVDYYYYGMDVWGQGTTVTVSS
CH65 Light chain lambda
QSVLTQPPSVSVAPGQTARITCGGNDIGRKSVHWNQQKPGQAPVLVVCYD
SDRPSGIPERFSGSNSGNTATLTISRVEAGDEADYYCQVWDSSSDHVIFG
GGTKLTVL

| | | |
|---|---|---|
| | Residue | |
| | numbers | Sequence (Kabat insertions |
| region | (Kabat) | in bold, deletions underscored) |

CH65 heavy chain

| | | |
|---|---|---|
| FR1 | 1-25 | EVQLVQSGAEVKKPGASVKVSCKAS |
| CDR1 | 26-35 | GYTFTDYHIN |
| FR2 | 36-49 | WVRQAPGQGLEWMG |
| CDR2 | 50-58 | WIHPNSGDTN |
| FR3 | 59-94 | YAQKFQGWVTMTRDTAISTAYMEVNGLKSDDTAVYY |
| | | CAR |
| CDR3 | 95-102 | GGLEPRSVDYYYYGMDV |
| FR4 | 103-113 | WGQGTTVTVSS |

CH65 light chain

| | | |
|---|---|---|
| FR1 | 1-23 | QSVLTQPPSVSVAPGQTARIT_C |
| CDR1 | 24-34 | GGNDIGRKSVH |
| FR2 | 35-49 | WNQQKPGQAPVLVVC |
| CDR2 | 50-56 | YDSDRPS |
| FR3 | 57-88 | GIPERFSGSNSGNTATLTISRVEAGDEADYYC |
| CDR3 | 89-97 | QVWDSSSDHVI |
| FR4 | 98-107 | FGGGTKLTVL |

5J8 Heavy Chain
EVQLVESGPGLVKPSDILSLTCAVSGYSISSNYYWGWIRQPPGKGLEWIG
SIYHSGSTYYKPSLESRLGISVDTSKNQFSLKLSFVSAADTAVYYCARHV
RSGYPDTAYYFDKWGQGTLVTVSs
5J8 Light Chain lambda
TSYVLTQPPSVSVAPGETARISCGGNNIGTKVLHWYQQTPGQAPVLVVYD
DSDRPSGIPERFSGSNSGNTATLTISRVEVGDEADYYCQVWDISTDQAVF
GGGTKLTVL

| | | |
|---|---|---|
| | Residue | |
| | numbers | Sequence (Kabat insertions |
| region | (Kabat) | in bold, deletions underscored) |

5J8 heavy chain

| | | |
|---|---|---|
| FR1 | 1-25 | EVQLVESGPGLVKPSDILSLTCAVS |
| CDR1 | 26-35 | GYSISSNYYWG |
| FR2 | 36-49 | WIRQPPGKGLEWIG |
| CDR2 | 50-58 | SIYHSGSTY |
| FR3 | 59-94 | YKPSLESRLGISVDTSKNQFSLKLSFVSAADTAVYY |
| | | CAR |
| CDR3 | 95-102 | HVRSGYPDTAYYFDK |
| FR4 | 103-113 | WGQGTLVTVSS |

5J8 light chain

| | | |
|---|---|---|
| FR1 | 1-23 | TSYVLTQPPSVSVAPGETARISC |
| CDR1 | 24-34 | GGNNIGTKVLH |
| FR2 | 35-49 | WYQQTPGQAPVLVVY |
| CDR2 | 50-56 | DDSDRPS |
| FR3 | 57-88 | GIPERFSGSNSGNTATLTISRVEVGDEADYYC |
| CDR3 | 89-97 | QVWDISTDQAV |
| FR4 | 98-107 | FGGGTKLTVL |

4K8 Heavy Chain
EVQLVESGGGLVQPGGSLRLSCAASEFNFKSYWMTWVRQAPGKGLEWVAN
INQDGSEKNYVDSVKGRFTISRDNAKNSLHLQMSSLRVDDTAVYYCARTG
SSWDTYYYYYAMDVWGQGTTVTVSS
4K8 Light Chain
DIQLTQSPVSLSLSPGERATLSCRASQSVSSSYLVWYQQKPGQAPRLLIY
GASSRAPGIPDRFSGSGSGTDFTLTISRLEREDFAVYYCQQYGRSFGQGT
KVEIK

| | | |
|---|---|---|
| | Residue | |
| | numbers | Sequence (Kabat insertions |
| region | (Kabat) | in bold, deletions underscored) |

4K8 heavy chain

| | | |
|---|---|---|
| FR1 | 1-25 | EVQLVESGGGLVQPGGSLRLSCAAS |
| CDR1 | 26-35 | EFNFKSYWMT |
| FR2 | 36-49 | WVRQAPGKGLEWVA |
| CDR2 | 50-58 | NINQDGSEKN |
| FR3 | 59-94 | YVDSVKGRFTISRDNAKNSLHLQMSSLRVDDTAVYY |
| | | CAR |
| CDR3 | 95-102 | TGSSWDTYYYYYAMDV |
| FR4 | 103-113 | WGQGTTVTVSS |

4K8 heavy chain

| | | |
|---|---|---|
| FR1 | 1-23 | DIQLTQSPVSLSLSPGERATLSC |
| CDR1 | 24-34 | RASQSVSSSYLV |
| FR2 | 35-49 | WYQQKPGQAPRLLIY |
| CDR2 | 50-56 | GASSRAP |
| FR3 | 57-88 | GIPDRFSGSGSGTDFTLTISRLEREDFAVYYC |
| CDR3 | 89-97 | QQYGRSFGQ |
| FR4 | 98-104 | GTKVEIK |

C179 Heavy chain
EVKLVESGGGLVQPGGSLRLSCGTSGFTLTDDYMTWVRQPPGKALEWLGF
IRDRANGYTTEYSASVKGRFTISRDNSQSIVYLQMNTLRVEDSATYYCAR
PKGYFPYAMDYWGQGTSVIVSS
C179 Light chain lambda
DIQMTQSPASQSASLGESVTITCLASQTIGTWLAWYQQKPGKSPQLLIYA
ATSLADGVPSRFSGSGSGTKFSFKISSLQAEDFVSYYCQQLYSTPWTFGG
GTRLEIK

| | | |
|---|---|---|
| | Residue | |
| | numbers | Sequence (Kabat insertions in |
| region | (Kabat) | bold, deletions underscored) |

C179 heavy chain

| | | |
|---|---|---|
| FR1 | 1-25 | EVKLVESGGGLVQPGGSLRLSCGTS |
| CDR1 | 26-35 | GFTLTDDYMT |
| FR2 | 36-49 | WVRQPPGKALEWLG |
| CDR2 | 50-58 | FIRDRANGYTTE |
| FR3 | 59-94 | YSASVKGRFTISRDNSQSIVYLQMNTLRVEDSATYY |
| | | CAR |
| CDR3 | 95-102 | PKGYFPYAMDY |
| FR4 | 103-113 | WGQGTSVIVSS |

C179 light chain

| | | |
|---|---|---|
| FR1 | 1-23 | DIQMTQSPASQSASLGESVTITC |
| CDR1 | 24-34 | LASQTIGTWLA |
| FR2 | 35-49 | WYQQKPGKSPQLLIY |
| CDR2 | 50-56 | AATSLAD |
| FR3 | 57-88 | GVPSRFSGSGSGTKFSFKISSLQAEDFVSYYC |
| CDR3 | 89-97 | QQLYSTPWT |
| FR4 | 98-107 | FGGGTRLEIK |

CR6261 Heavy chain
EVQLVESGAEVKKPGSSVKVSCKASGGPFRSYAISWVRQAPGQGPEWMGG
IIPIFGTTKYAPKFQGRVTITADDFAGTVYMELSSLRSEDTAMYYCAKHM
GYQVRETMDVWGKGTTVTVSS
CR6261 Light chain
QSVLTQPPSVSAAPGQKVTISCSGSSSNIGNDYVSWYQQLPGTAPKLLIY
DNNKRPSGIPDRFSGSKSGTSATLGITGLQTGDEANYYCATWDRRPTAYV
VFGGGTKLTVL

| | | |
|---|---|---|
| | Residue | |
| | numbers | Sequence (Kabat insertions in |
| region | (Kabat) | bold, deletions underscored) |

CR6261 heavy chain

| | | |
|---|---|---|
| FR1 | 1-25 | EVQLVESGAEVKKPGSSVKVSCKAS |
| CDR1 | 26-35 | GGPFRSYAIS |
| FR2 | 36-49 | WVRQAPGQGPEWMG |
| CDR2 | 50-58 | GIIPIFGTTK |
| FR3 | 59-94 | YAPKFQGRVTITADDFAGTVYMELSSLRSEDTAMYY |
| | | CAK |
| CDR3 | 95-102 | HMGYQVRETMDV |
| FR4 | 103-113 | WGKGTTVTVSS |

CR6261 light chain

| | | |
|---|---|---|
| FR1 | 1-23 | QSVLTQPPSVSAAPGQKVTIS_C |
| CDR1 | 24-34 | SGSSSNIGNDYVS |
| FR2 | 35-49 | WYQQLPGTAPKLLIY |
| CDR2 | 50-56 | DNNKRPS |
| FR3 | 57-88 | GIPDRFSGSKSGTSATLGITGLQTGDEANYYC |
| CDR3 | 89-97 | ATWDRRPTAYVV |
| FR4 | 98-107 | FGGGTKLTVL |

FI6 Heavy chain
QVQLVESGGGVVQPGRSLRLSCAASGFTFSTYAMHWVRQAPGKGLEWVAV
ISYDANYKYYADSVKGRFTISRDNSKNTLYLQMNSLRAEDTAVYYCAKDS
QLRSLLYFEWLSQGYFDYWGQGTLVTVSS
FI6 Light chain
DIVMTQSPDSLAVSLGERATINCKSSQSVTFNYKNYLAWYQQKPGQPPKL
LIYWASTRESGVPDRFSGSGSGTDFTLTISSLQAEDVAVYYCQQHYRTPP
TFGQGTKVEIK

| | | |
|---|---|---|
| | Residue | |
| | numbers | Sequence (Kabat insertions in |
| region | (Kabat) | bold, deletions underscored) |

FI6 heavy chain

| | | |
|---|---|---|
| FR1 | 1-25 | QVQLVESGGGVVQPGRSLRLSCAAS |
| CDR1 | 26-35 | GFTFSTYAMH |
| FR2 | 36-49 | WVRQAPGKGLEWVA |
| CDR2 | 50-58 | VISYDANYK |
| FR3 | 59-94 | YYADSVKGRFTISRDNSKNTLYLQMNSLRAEDTAVY |
| | | YCAK |
| CDR3 | 95-102 | DSQLRSLLYFEWLSQGYFDY |
| FR4 | 103-113 | WGQGTLVTVSS |

FI6 light chain

| | | |
|---|---|---|
| FR1 | 1-23 | DIVMTQSPDSLAVSLGERATINC |
| CDR1 | 24-34 | KSSQSVTFNYKNYLA |
| FR2 | 35-49 | WYQQKPGQPPKLLIY |
| CDR2 | 50-56 | WASTRES |
| FR3 | 57-88 | GVPDRFSGSGSGTDFTLTISSLQAEDVAVYYC |
| CDR3 | 89-97 | QQHYRTPPT |
| FR4 | 98-107 | FGQGTKVEIK |

F10 Heavy chain
QVQLVQSGAEVKKPGSSVKVSCTSSEVTFSSFAISWVRQAPGQGLEWLGG
ISPMFGTPNYAQKFQGRVTITADQSTRTAYMDLRSLRSEDTAVYYCARSP
SYICSGGTCVFDHWGQGTLVTVSS
F10 Light chain
IQPGLTQPPSVSKGLRQTATLTCTGNSNNVGNQGAAWLQQHQGHPPKLLS
YRNNDRPSGISERFSASRSGNTASLTITGLQPEDEADYYCSTWDSSLSAV
VFGGGTKLTVLGQPKAAPSAA

| | | |
|---|---|---|
| | Residue | |
| | numbers | Sequence (Kabat insertions in |
| region | (Kabat) | bold, deletions underscored) |

F10 heavy chain

| | | |
|---|---|---|
| FR1 | 1-25 | QVQLVQSGAEVKKPGSSVKVSCTSS |
| CDR1 | 26-35 | EVTFSSFAIS |
| FR2 | 36-49 | WVRQAPGQGLEWLG |
| CDR2 | 50-58 | GISPMFGTPN |
| FR3 | 59-94 | YAQKFQGRVTITADQSTRTAYMDLRSLRSEDTAVY |
| | | YCAR |
| CDR3 | 95-102 | SPSYICSGGTCVFDH |
| FR4 | 103-113 | WGQGTLVTVSS |

F10 light chain

| | | |
|---|---|---|
| FR1 | 1-23 | IQPGLTQPPSVSKGLRQTATLTC |
| CDR1 | 24-34 | TGNSNNVGNQGAA |
| FR2 | 35-49 | WLQQHQGHPPKLLSY |
| CDR2 | 50-56 | RNNDRPS |
| FR3 | 57-88 | GISERFSASRSGNTASLTITGLQPEDEADYYC |
| CDR3 | 89-97 | STWDSSLSAVV |
| FR4 | 98-107 | FGGGTKLTVL |

In some embodiments, suitable neutralizing antibodies for the present invention may include various antibodies that have similar or substantially identical binding specificity as those described above (e.g., CH65, 5J8, 4K8, FI6, C179, F10, or CR6261). For example, suitable neutralizing antibodies may include antibodies that are capable of competing with any of the antibodies described above (e.g., CH65, 5J8, 4K8, FI6, C179, F10, or CR6261). In some embodiments, suitable neutralizing antibodies for the present invention may include antibodies that contain a light chain and/or a heavy chain that contains one or more amino acid or domain substitutions, deletions, and/or insertions as compared to the light chain and/or heavy chain of any of the antibodies described above (e.g., CH65, 5J8, FI6, C179, F10, or CR6261), while retaining substantial binding specificity. Thus, in some embodiments, suitable neutralizing antibodies for the present invention may include antibodies that contain a heavy chain substantially homologous or identical to the heavy chain of any of the antibodies described above (e.g., CH65, 5J8, 4K8, FI6, C179, F10, or CR6261). In some embodiments, suitable neutralizing antibodies for the present invention may include antibodies that contain a heavy chain having an amino acid sequence at least 50%, 55%, 60%, 65%, 70%, 75%, 80%, 85%, 90%, 91%, 92%, 93%, 94%, 95%, 96%, 97%, 98%, 99% or more homologous or identical to the heavy chain of any of the antibodies described above (e.g., CH65, 5J8, 4K8, FI6, C179, F10, or CR6261). In some embodiments, suitable neutralizing antibodies for the present invention may include antibodies that contain a light chain substantially homologous or identical to the light chain of any of the antibodies described above (e.g., CH65, 5J8, 4K8, FI6, C179, F10, or CR6261). In some embodiments, suitable neutralizing antibodies for the present invention may include antibodies that contain a light chain having an amino acid sequence at least 50%, 55%, 60%, 65%, 70%, 75%, 80%, 85%, 90%, 91%, 92%, 93%, 94%, 95%, 96%, 97%, 98%, 99% or more homologous or identical to the light chain of any of the antibodies described above (e.g., CH65, 5J8, 4K8, FI6, C179, F10, or CR6261).

In some embodiments, suitable neutralizing antibodies for the present invention may include antibodies that contain one or more heavy chain and/or light chain CDRs as described herein. For example, a neutralizing antibody against HA stem may be defined by: a heavy chain CDR1 sequence selected from the group consisting of GFTFSTYAMH, GFTLTDDYMT, GGPFRSYAIS, and EVTFSSFAIS; a heavy chain CDR2 sequence selected from the group consisting of VISYDANYK, FIRDRANGYTTE, GIIPIFGTTK, and GISPMFGTPN; a heavy chain CDR3 sequence selected from the group consisting of DSQLRSLLYFEWLSQGYFDY, PKGYFPYAMDY, HMGYQVRETMDV, and SPSYICSGGTCVFDH; a light chain CDR1 sequence selected from the group consisting of KSSQSVTFNYKNYLA, LASQTIGTWLA, SGSSSNIGNDYVS, and TGNSNNVGNQGAA; a light chain CDR2 sequence selected from the group consisting of WASTRES, AATSLAD, DNNKRPS, and RNNDRPS; or a light chain CDR3 sequence selected from the group consisting of QQHYRTPPT, QQLYSTPWT, ATWDRRPTAYVV, and STWDSSLSAVV.

In particular embodiments, a suitable neutralizing antibody against HA stem may be defined by:
a heavy chain comprising a CDR1 sequence of GFTLTDDYMT, CDR2 sequence of FIRDRANGYTTE, and CDR3 sequence of PKGYFPYAMDY; and a light chain comprising a CDR1 sequence of LASQTIGTWLA, CDR2 sequence of AATSLAD, and CDR3 sequence of QQLYSTPWT;
a heavy chain comprising a CDR1 sequence of GGPFRSYAIS, CDR2 sequence of GIIPIFGTTK, and CDR3 sequence of HMGYQVRETMDV; and a light chain comprising a CDR1 sequence of SGSSSNIGNDYVS, CDR2 sequence of DNNKRPS, and CDR3 sequence of ATWDRRPTAYVV; or
a heavy chain comprising a CDR1 sequence of EVTFSSFAIS, CDR2 sequence of GISPMFGTPN, and CDR3 sequence of SPSYICSGGTCVFDH; and a light chain comprising a CDR1 sequence of TGNSNNVGNQGAA, CDR2 sequence of RNNDRPS, and CDR3 sequence of STWDSSLSAVV; or
a heavy chain comprising a CDR1 sequence of GFTFSTYAMH, CDR2 sequence of VISYDANYK, and CDR3 sequence of DSQLRSLLYFEWLSQGYFDY; and a light chain comprising a CDR1 sequence of KSSQSVTFNYKNYLA, CDR2 sequence of WASTRES, and CDR3 sequence of QQHYRTPPT.

In some embodiments, a suitable neutralizing antibody against HA head may be defined by: a heavy chain CDR1 sequence selected from the group consisting of GYTFTDYHIN, GYSISSNYYWG, and EFNFKSYWMT; a heavy chain CDR2 sequence selected from the group consisting of WIHPNSGDTN, SIYHSGSTY, and NINQDGSEKN; a heavy chain CDR3 sequence selected from the group consisting of GGLEPRSVDYYYYGMDV, HVRSGYPDTAYYFDK and TGSSWDTYYYYYAMDV; a light chain CDR1 sequence selected from the group consisting of GGNDIGRKSVH, GGNNIGTKVLH, and RASQSVSSSYLV; a light chain CDR2 sequence selected from the group consisting of YDSDRPS, DDSDRPS, and GASSRAP; or a light chain CDR3 sequence selected from the group consisting of QVWDSSSDHVI, QVWDISTDQAV, and QQYGRSFGQ.

In particular embodiments, a suitable neutralizing antibody against HA head may be defined by:
a heavy chain comprising a CDR1 sequence of GYTFTDYHIN, CDR2 sequence of WIHPNSGDTN, and CDR3 sequence of GGLEPRSVDYYYYGMDV; and a light chain comprising a CDR1 sequence of GGNDIGRKSVH, CDR2 sequence of YDSDRPS, and CDR3 sequence of QVWDSSSDHVI;
a heavy chain comprising a CDR1 sequence of GYSISSNYYWG, CDR2 sequence of SIYHSGSTY, and CDR3 sequence of HVRSGYPDTAYYFDK; and a light chain comprising a CDR1 sequence of GGNNIGTKVLH, CDR2 sequence of DDSDRPS, and CDR3 sequence of QVWDISTDQAV; or
a heavy chain comprising a CDR1 sequence of EFNFKSYWMT, CDR2 sequence of NINQDGSEKN, and CDR3 sequence of TGSSWDTYYYYYAMDV; and a light chain comprising a CDR1 sequence of RASQSVSSSYLV, CDR2 sequence of GASSRAP, and CDR3 sequence of QQYGRSFGQ.

Suitable neutralizing antibodies may be directly generated from various host animals including, but not limited to, humans, non-human primates, mice, rats, rabbits, monkeys, dogs, cats, sheeps, goats, cattles, pigs, and lama. Suitable neutralizing antibodies may also be chimeric antibodies with CDRs (such as those described herein) grafted into framework regions derived from antibodies generated from various animals described herein.

### Immunostaining and Detection of Binding Levels

Engineered HA antigen displayed on the surface of cells may be stained with a panel of neutralizing antibodies described herein using various methods known in the art. In particular, immunostaining of the cells with a panel of neutralizing antibodies described herein is performed under conditions that permit the neutralizing antibodies to bind to the engineered HA antigens displayed on the surface of the cells, but not to the HA antigens expressed inside the cells. In some such embodiments, the integrity of the cell membrane is preserved such that antibodies are not able to penetrate the cell membrane. Exemplary methods for cell surface HA staining are described in the Example sections and additional methods are available in the art and can be used to practice the present invention.

To facilitate detection of binding between neutralizing antibodies and engineered HA antigens displayed on the cell surface, the neutralizing antibodies may be labeled with a detectable entity that generates a detectable signal. In some embodiments, secondary antibodies recognizing the neutralizing antibodies are used to detect the binding. In that case, the secondary antibodies are typically labeled with a detectable entity that generates a detectable signal.

Detectable Entities

Any of a wide variety of detectable agents can be used in the practice of the present invention. Suitable detectable entities include, but are not limited to: fluorescent dyes; chemiluminescent agents (such as, for example, acridinum esters, stabilized dioxetanes, and the like); bioluminescent agents; enzymes; colorimetric labels (such as, for example, dyes, colloidal gold, and the like); biotin; dioxigenin; haptens; and proteins for which antisera or monoclonal antibodies are available.

In certain embodiments, a detectable moiety is a fluorescent dye. Numerous known fluorescent dyes of a wide variety of chemical structures and physical characteristics are suitable for use in the practice of the present invention. Suitable fluorescent dyes include, but are not limited to, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514., etc.), Texas Red, Texas Red-X, SPECTRUM RED™, SPECTRUM GREEN™, cyanine dyes (e.g., CY-3™, CY-5™, CY-3.5™, CY-5.5™, etc.), ALEXA FLUOR™ dyes (e.g., ALEXA FLUOR™ 350, ALEXA FLUOR™ 488, ALEXA FLUOR™ 532, ALEXA FLUOR™ 546, ALEXA FLUOR™ 568, ALEXA FLUOR™ 594, ALEXA FLUOR™ 633, ALEXA FLUOR™ 660, ALEXA FLUOR™ 680, etc.), BODIPY™ dyes (e.g., BODIPY™ FL, BODIPY™ R6G, BODIPY™ TMR, BODIPY™ TR, BODIPY™ 530/550, BODIPY™ 558/568, BODIPY™ 564/570, BODIPY™ 576/589, BODIPY™ 581/591, BODIPY™ 630/650, BODIPY™ 650/665, etc.), IRDyes (e.g., IRD40, IRD 700, IRD 800, etc.), and the like. For more examples of suitable fluorescent dyes and methods for coupling fluorescent dyes to other chemical entities such as proteins and peptides, see, for example, “The Handbook of Fluorescent Probes and Research Products”, 9th Ed., Molecular Probes, Inc., Eugene, Oreg. Favorable properties of fluorescent labeling agents include high molar absorption coefficient, high fluorescence quantum yield, and photostability. In some embodiments, labeling fluorophores exhibit absorption and emission wavelengths in the visible (i.e., between 400 and 750 nm) rather than in the ultraviolet range of the spectrum (i.e., lower than 400 nm).

Flow Cytometry Analysis

Various methods may be used to detect bindings between neutralizing antibodies and engineered HA antigens displayed on the cell surface. In some embodiments, the bindings on the cell surface are detected through the use of flow cytometry. Flow cytometry is a laser-based, biophysical technology employed in cell counting, cell sorting, biomarker detection and protein engineering, by suspending cells in a stream of fluid and passing them by an electronic detection apparatus. Scanning for multiple parameters, flow cytometry allows simultaneous analysis of the physical and chemical characteristics of up to thousands of cells per second. Flow cytometers can analyze several thousand cells every second and can actively separate and sort cells based on the detectable signals associated with the neutralizing antibodies bound to the HA antigens displayed on the cell surface. Thus, a flow cytometer offers “high-throughput” (for a large number of cells), automated quantification of set parameters.

The data generated by flow-cytometers can be plotted in a single dimension (to produce a histogram), in two-dimensional dot plots or in three dimensions. The regions on these plots can be sequentially separated, based on fluorescence intensity, by creating a series of subset extractions, termed “gates.” Plots are often made on logarithmic scales. Because different fluorescent dyes' emission spectra overlap, signals at the detectors have to be compensated computationally and electronically. Data accumulated using the flow cytometer can be analyzed using software, e.g., WinMDI, Flowing Software, and web-based Cytobank (all freeware), FCS Express, Flowjo, FACSDiva, CytoPaint (aka Paint-A-Gate), VenturiOne, CellQuest Pro, Infinicyt or Cytospec.

In some embodiments, flow cytometry data is in the form of a large matrix of M intensities by N events. Most events will be a particular cell, although some may be doublets (pairs of cells which pass the laser closely together). For each event, the measured fluorescence intensity over a particular wavelength range is recorded. The measured fluorescence intensity indicates the amount of that fluorophore in the cell, which indicates the binding levels between the neutralizing antibodies and HA antigens displayed on the surface of the cells. In some embodiments, flow cytometry data can be considered a matrix of M measurements of amounts of molecules of interest by N cells. Averages of the M measurements offset by background may be used to calculate antibody binding levels. In some embodiments, median fluorescence intensity (MFI) of a population of N cells (e.g., about 100 cells, 1,000 cells, 5,000 cells, 10,000 cells, 15,000 cells, 20,000 cells or more) is used to quantitatively calculate antibody binding levels. Typically, the binding levels are quantitatively determined as compared to a reference level or a benchmark (such as, a wild-type benchmark or background). A wild-type benchmark is typically defined by cells expressing a wild-type HA antigen that is used as a template for HA engineering. A background level is typically set by mock-transfected or transformed cells (i.e., cells that do not contain a nucleic acid encoding an engineered HA antigen). Exemplary methods for detecting and calculating binding levels between naturalizing antibodies and HA antigens are described in the Examples sections. Additional methods are known in the art and can be used to practice the present invention.

Down-Selection of Engineered HA Antigens

Typically, the binding levels measured using anti-HA stem neutralizing antibodies may be used to assess expression and conformation of an engineered HA antigen and the binding levels measured using anti-HA head neutralizing antibodies may be used to assess conformation. In some embodiments, an engineered antigen is down-selected as properly expressed if the binding levels to HA antigen are 50% or higher (e.g., 50%, 55%, 60%, 65%, 70%, 75%, 80%, 85%, 90%, 95%, 1-fold, 1.5-fold, or 2-fold) compared to a wild-type benchmark for at least 2, 3, 4, 5, 6, or more neutralizing antibodies against HA stem.

In some embodiments, an engineered HA antigen is down-selected as properly folded if the binding levels are above background (e.g., at least 1-fold, 1.5-fold, 2-fold, 2.5-fold, 3-fold, 4-fold, or 5-fold higher) for at least one neutralizing antibody against HA head and at least 2, 3, 4, 5, 6, or all neutralizing antibodies tested against HA stem.

Engineered HA antigens can also be down-selected to facilitate generation of broadly neutralizing antibodies against particular specific strains, for example a pandemic or seasonal influenza strain. This can be accomplished by including in the panel one or more appropriate antibodies that will bind to and select a conformationally accurate epitope only for the specific strain of interest. In other words, the antibodies comprising the antibody panel can be customized to a specified antibody binding pattern (e.g., seasonal or pandemic) that selects HA antigens that will elicit neutralizing antibody responses of particular therapeutic interest against a targeted strain. The customizability of the assays described herein is limited only by the availability of suitable antibodies against the targeted strain.

### Immunogenicity Analysis

Down-selected HA antigens may be tested for their ability to elicit neutralizing antibody response using in vitro or in vivo methods. Various immunogenicity analysis methods are well known in the art and can be used to practice the invention. Typically, down-selected HA antigens are produced as part of viral-like particles (VLPs) or other vaccine compositions (such as live attenuated virus, split virus, or purified recombinant HA polypeptides) and injected into animals to determine if the down-selected HA antigens can induce neutralizing antibody response in vivo. Animal hosts suitable for the invention can be any mammalian hosts, including primates, ferrets, cats, dogs, cows, horses, rodents such as, mice, hamsters, rabbits, and rats. In some embodiments, an animal host used for the invention is a ferret. In particular, in some embodiments, an animal host is naïve to viral exposure or infection prior to administration of a binding agent in accordance with the invention (optionally in a composition in accordance with the invention). In some embodiments, the animal host is inoculated with, infected with, or otherwise exposed to virus prior to or concurrent with administration of an engineered HA polypeptide screened in accordance with the invention. An animal host used in the practice of the present invention can be inoculated with, infected with, or otherwise exposed to virus by any method known in the art.

Various assays may be used to measure neutralizing antibody responses induced by down-selected HA antigens. For example, hemagglutination inhibition assay with immune sera obtained from injected host animals may be used to measure neutralizing antibody responses. As shown in the Examples section, down-selected HA antigens using a screen assay according to the present invention are able to induce neutralizing antibody responses. In some embodiments, down-selected HA antigens according to the present invention are able to induce broadly neutralizing antibodies against multiple (e.g., at least 2, 3, 4, 5, 6, 7, 8, 9, 10, or more) distinct influenza strains including pandemic, pre-pandemic, historical and/or contemporary strains. In addition, the present invention may be used to predict, assess and/or validate specificity against strain clusters based on anti-head antibody binding analysis as described herein.

The present invention may be used to down-select or validate any engineered HA antigens for manufacturing any form of influenza vaccine including monovalent, divalent, trivalent and quadrivalent formulations.

### Kits

The present invention further provides kits for performing various screening assays described herein. In particular, the present invention provides kits including a panel of neutralizing antibodies as described herein for immunostaining of HA antigens. In some embodiments, the kits can further include reagents for immunostaining and/or reagents for use in flow cytometry analyses. For example, kits for use in accordance with the present invention may include, a reference sample, instructions for processing samples, performing the test, instructions for interpreting the results, buffers and/or other reagents necessary for performing the test.

Although the present invention and its advantages have been described in detail, it should be understood that various changes, substitutions and alterations can be made herein without departing from the spirit and scope of the invention as defined in the appended claims.

The invention will be more fully understood by reference to the following examples. They should not, however, be construed as limiting the scope of the invention. All literature citations are incorporated by reference.

### EXAMPLES

These Examples are set forth to aid in the understanding of the invention but are not intended to, and should not be construed to, limit its scope in any way. The Examples do not include detailed descriptions of conventional methods that would be well known to those of ordinary skill in the art (molecular cloning techniques, etc.). Unless indicated otherwise, parts are parts by weight, molecular weight is average molecular weight, temperature is indicated in Celsius, and pressure is at or near atmospheric.

### Example 1

### Rapid Screening of Engineered HA Antigens

Vaccine-mediated protection is conferred by the neutralizing antibody response against hemagglutinin (HA). However, accumulation of genetic mutations in the HA gene gives rise to new strains that can evade neutralization, which is also known as antigenic drift. Therefore, current flu vaccines must be periodically revised to adjust for such changes in the HA antigen. Ideal flu vaccines should be able to stimulate broad neutralizing antibody responses that are effective against circulating strains and against drifted/mismatched strains. Such vaccines are also known as universal influenza vaccines. Universal influenza vaccines can be rationally designed by re-engineering HA antigens to elicit broadly cross-neutralizing antibodies. However, a robust in vitro screening assay is needed to identify and focus on most promising in silico designs for pre-clinical studies.

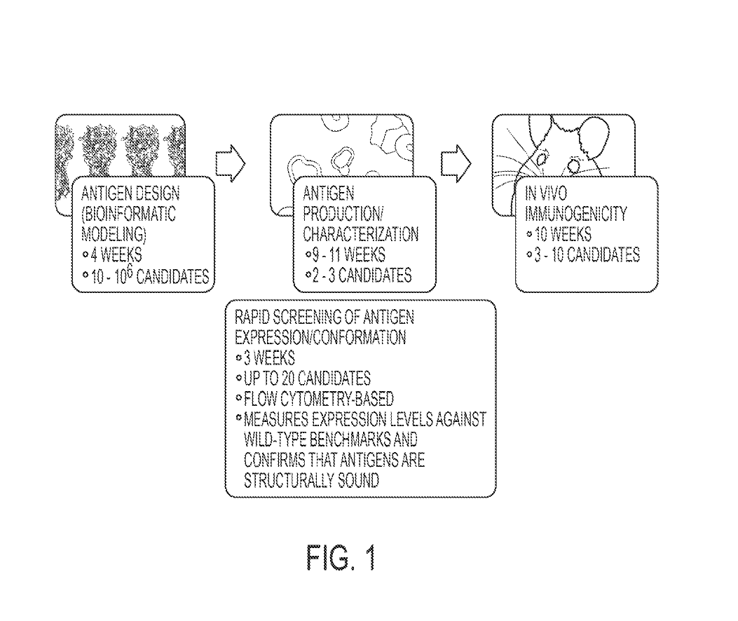

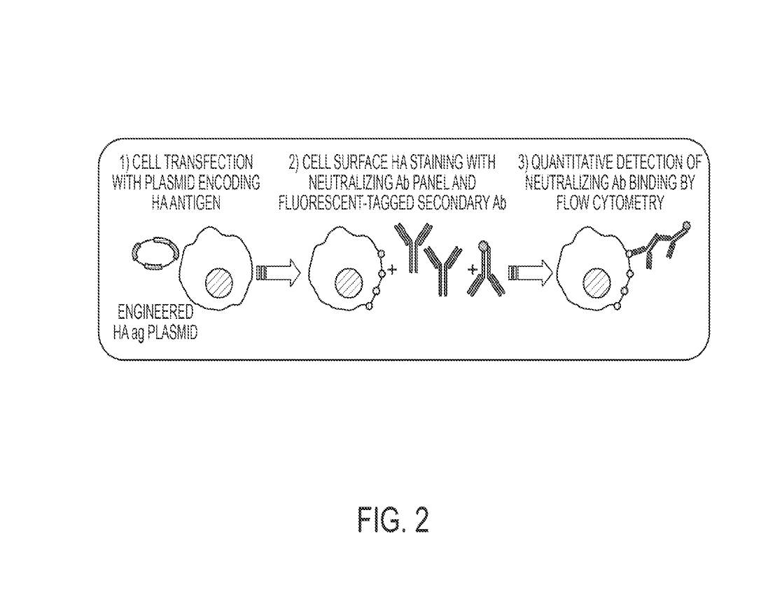

This example outlines the steps for such an in vitro screening assay that rapidly identifies promising flu vaccine candidates for pre-clinical studies. Representative steps are shown in FIG. 1. Conventional screening methods characterize HA expression using western-blot, which requires protein purification. Typically, it takes about 3 weeks to characterize just 2-3 candidates, which brings total antigen production time to 9-11 weeks. By contrast, this new approach can screen up to 20 candidates within about 3 weeks, and without protein purification as the transfected cells provide the HA antigens for analysis. Briefly, the present method takes advantage of the flow cytometry technology to measure binding of neutralizing antibodies to HA antigens expressed on the surface of transfected cells (FIG. 2). In addition, since the fluorescence intensity is proportional to antibody binding and antigen expression, this method provides compartmentalized and quantitative characterization of expression and/or conformation of only surface displayed HA antigens and allows identification of those engineered HA antigens that can be robustly expressed and structurally sound.

### Example 2

### Panel of Neutralizing Antibodies for Screening

This example illustrates an exemplary panel of neutralizing antibodies for screening of engineered HA antigens. Typically, broadly neutralizing antibodies against HA antigens were used. FIG. 3 depicts a tertiary structure of an HA antigen. As shown, HA is a homotrimeric glycoprotein. It is shaped like a mushroom and can be generally divided into the head and stem regions. The head region contains the receptor binding site that recognizes sialic acid. It is contemplated that a panel of antibodies suitable for screening typically includes one or more neutralizing antibodies against HA head, in particular, those antibodies that bind to epitopes close to the receptor binding site, and one or more neutralizing antibodies against HA stem, in particular, those antibodies that bind highly conversed conformational epitopes of the stem.

The exemplary panel described in this example includes 3 anti-head neutralizing antibodies: CH65 (contemporary strains prior 2009 pandemic) (Whittle, JRR, et al. PNAS 2011), 5J8 (contemporary and historical strains) (Krause, J C, et al. J. Virology 2011), and 4K8 (pandemic strains only) (Krause, J C, et al., J. Immunology 2011); and 3 anti-stem neutralizing antibodies: C179 (group 1 HAs) (Okun, Y et al., J. Virology 1993), F10 (group 1 HAs) (Sui, et al., Nature Struct. & Mol. Bio, 2009), and CR6261 (group 1 HAs) (Ekiert et al., Science, 2009). The amino acid sequences of antibodies CH65, 5J8, 4K8, C179, F10 and CR6261 are shown below.

C179 Heavy chain
EVKLVESGGGLVQPGGSLRLSCGTSGFTLTDDYMTWVRQPPGKALEWLGF
IRDRANGYTTEYSASVKGRFTISRDNSQSIVYLQMNTLRVEDSATYYCAR
PKGYFPYAMDYWGQGTSVIVSS
C179 Light chain lambda
DIQMTQSPASQSASLGESVTITCLASQTIGTWLAWYQQKPGKSPQLLIYA
ATSLADGVPSRFSGSGSGTKFSFKISSLQAEDFVSYYCQQLYSTPWTFGG
GTRLEIK
CR6261 Heavy chain
EVQLVESGAEVKKPGSSVKVSCKASGGPFRSYAISWVRQAPGQGPEWMGG
IIPIFGTTKYAPKFQGRVTITADDFAGTVYMELSSLRSEDTAMYYCAKHM
GYQVRETMDVWGKGTTVTVSS
CR6261 Light chain
QSVLTQPPSVSAAPGQKVTISCSGSSSNIGNDYVSWYQQLPGTAPKLLIY
DNNKRPSGIPDRFSGSKSGTSATLGITGLQTGDEANYYCATWDRRPTAYV
VFGGGTKLTVL
F10 Heavy chain
QVQLVQSGAEVKKPGSSVKVSCTSSEVTFSSFAISWVRQAPGQGLEWLGG
ISPMFGTPNYAQKFQGRVTITADQSTRTAYMDLRSLRSEDTAVYYCARSP
SYICSGGTCVFDHWGQGTLVTVSS
F10 Light chain
IQPGLTQPPSVSKGLRQTATLTCTGNSNNVGNQGAAWLQQHQGHPPKLLS
YRNNDRPSGISERFSASRSGNTASLTITGLQPEDEADYYCSTWDSSLSAV
VFGGGTKLTVLGQPKAAPSAA
CH65 Heavy chain
EVQLVQSGAEVKKPGASVKVSCKASGYTFTDYHINWVRQAPGQGLEWMGW
IHPNSGDTNYAQKFQGWVTMTRDTAISTAYMEVNGLKSDDTAVYYCARGG
LEPRSVDYYYYGMDVWGQGTTVTVSS
CH65 Light chain lambda
QSVLTQPPSVSVAPGQTARITCGGNDIGRKSVHWNQQKPGQAPVLVVCYD
SDRPSGIPERFSGSNSGNTATLTISRVEAGDEADYYCQVWDSSSDHVIFG
GGTKLTVL
5J8 Heavy chain
EVQLVESGPGLVKPSDILSLTCAVSGYSISSNYYWGWIRQPPGKGLEWIG
SIYHSGSTYYKPSLESRLGISVDTSKNQFSLKLSFVSAADTAVYYCARHV
RSGYPDTAYYFDKWGQGTLVTVSs
5J8 Light chain lambda
TSYVLTQPPSVSVAPGETARISCGGNNIGTKVLHWYQQTPGQAPVLVVYD
DSDRPSGIPERFSGSNSGNTATLTISRVEVGDEADYYCQVWDISTDQAVF
GGGTKLTVL
4K8 Heavy chain
EVQLVESGGGLVQPGGSLRLSCAASEFNFKSYWMTWVRQAPGKGLEWVAN
INQDGSEKNYVDSVKGRFTISRDNAKNSLHLQMSSLRVDDTAVYYCARTG
SSWDTYYYYYAMDVWGQGTTVTVSS
4K8 Light chain
DIQLTQSPVSLSLSPGERATLSCRASQSVSSSYLVWYQQKPGQAPRLLIY
GASSRAPGIPDRFSGSGSGTDFTLTISRLEREDFAVYYCQQYGRSFGQGT
KVEIK

### Example 3

### Assay Conditions

This example illustrate exemplary assay conditions for rapid screening of engineered HA antigens as outlined in Example 1 and using the panel of neutralizing antibodies shown in Example 2.

### Plasmid Generation

Specifically, engineered HA amino acid sequences were back-translated, optimized for mammalian protein expression and the resulting nucleotide sequence was cloned into a mammalian expression vector . Plasmids encoding wild-type HA sequences used as template for HA engineering were also generated to be used as expression benchmarks.

Cell Transfection with Plasmids Encoding Engineered HA Antigens

Approximately 1×10⁶ HEK293FT cells were seeded into 12-well plates in 1 ml DMEM supplemented with 10% FBS. After overnight incubation at 37° C. in 8% CO2, cells were 90% confluent and used immediately for transfection. Plasmid DNAs encoding a single HA nucleotide sequence were mixed with Lipofectamine 2000 at a 3:1 ratio (pDNA:Lipofectamine) following manufacturer's instructions. A total of 2 micrograms of plasmid DNA in complex with Lipofectamine 2000 were added to each well of confluent HEK293FT cells, and cells were further incubated for 24 hours at 37° C. in 8% CO2.

### Immunofluorescent Staining for Detection of Surface HA Antigens

Transfected HEK293FT cells were harvested at 24 hours post-transfection by gentle dissociation with TrypLE Express and washed twice with PBS. Single-cell suspensions were labeled with LIVE/DEAD® Fixable Far Red Dead Cell Stain Kit to determine viability of the cells prior to surface staining according to manufacturer's instructions. Labeled cells were washed and re-suspended in staining buffer (0.1% BSA in PBS). Approximately 2×10⁵ cells re-suspended in 200 microliters of staining buffer were stained with 0.4 micrograms of unlabeled neutralizing anti-hemagglutinin monoclonal antibody (see Example 2) for 20 min at 4° C. Stained cells were washed and re-suspended in 100 microliters of staining buffer containing 0.2 micrograms of Alexa Fluor® 488 Anti-Human or Anti-Mouse IgG secondary antibody (depending on primary antibody) and stained with secondary antibody for 20 min at 4° C. Finally, stained cells were re-suspended in fixation solution (1.75% formaldehyde in PBS) and stored for≦1 week at 4° C.

### Flow Cytometry Analysis

Fixed cells were washed and re-suspended in 200 microliters of PBS, and then transferred to deep-well 96-well plate for sample acquisition using a BD High-Throughput Sampler. Sample analysis was performed using a BD FACS Calibur flow cytometer equipped with a 488 nm laser (for Alexa Fluor® 488 excitation) and a 635 nm laser (for LIVE/DEAD far red dye excitation). A mock-transfected cell sample stained with Alexa Fluor® 488 secondary antibody but no primary antibody was used to determine optimal acquisition settings. In particular forward-scatter (FSC) amplification gain, side-scatter (SSC) voltage and FSC threshold were adjusted to display the HEK293FT cell population on scale and to exclude unwanted debris. Cell population was gated in the FSC vs SSC plot to further exclude debris. Fluorescence detector settings were also adjusted using mock-transfected cells stained only with secondary antibody. In particular FL1 detector (for detection of Alexa Fluor® 488 fluorescence) and FL4 detector (for detection of LIVE/DEAD far red dye fluorescence) voltages were adjusted to place fluorescence emission of the gated cell population in first log decade. Compensation adjustments were not required for this fluorophore combination as there is no spectral overlap between Alexa Fluor® 488 and LIVE/DEAD far red dye. All samples were acquired using same acquisition settings as the mock control. At least 10,000 cells within FSC vs SSC gate were counted for each sample and data was saved as FCS data files.

Data analysis was performed using FlowJo software. FCS data file corresponding to mock-transfected cells stained only with secondary antibody was used to create analysis gates. In particular, a gate including intact cell population was first drawn in the FSC vs SSC plot. This gated cell subset was then analyzed in separate plot displaying FL4 fluorescence intensity (LIVE/DEAD far red dye fluorescence) vs FSC. A new gate encompassing the cell population with low FL4 fluorescence intensity was created. This new cell subset corresponding to intact live cells was further analyzed in separate plot displaying FL1 fluorescence intensity (Alexa Fluor® 488 fluorescence) vs FSC. A new gate encompassing cells with positive FL1 fluorescence as defined by fluorescence values that leave 95% of the mock-transfected cells in the negative FL1 fraction was generated. All FCS files were analyzed using the same analysis gates. Median fluorescence intensity (MFI) of positive FL1 cell subset for each cell sample and staining was exported to excel file and used to calculate antibody binding ratio.

MFI of positive FL1 cell subset for each cell sample and staining was first corrected by subtracting background fluorescence corresponding to same cell sample stained with secondary antibody only. Specificity of the staining with each of the neutralizing anti-hemagglutinin monoclonal antibodies was confirmed by examining the background corrected MFI of mock-transfected cells (negative control) and the background corrected MFI of cells transfected with wild-type HA plasmid DNA (positive control). If MFI for controls fell within expected range of values, then antibody binding ratio for each engineered HA plasmid and neutralizing anti-HA monoclonal antibody was determined as follows:

$$Antibody binding ratio ( A B R ) = \frac{MFI ( HA x , primary ab y ) - MFI ( HA x , secondary ab only )}{\begin{matrix}MFI ( wild \text{-} type HA , primary ab y ) - \\ MFI ( wild \text{-} type HA , secondary ab only )\end{matrix}}$$

Down-selection of Engineered HA Antigens

Typically, down-selected engineered HA antigens meet two criteria (expression and conformation) for further analysis. To satisfy expression criteria, the Antibody Binding Ratio (ABR) of tested HA should be >0.5 for at least three individually tested neutralizing antibodies against HA stem. This means antibody binds to HA antigens at 50% or higher compared to wild-type benchmark (defined by original wild-type HA used for computer modeling) for at least three neutralizing antibodies against the HA stem. To satisfy conformational or folding criteria, the Antibody Binding Ratio of tested HA should be higher than ABR for non-transfected cells (background) for at least one neutralizing antibody against HA head and all tested neutralizing antibodies against HA stem.

### Example 4

### Down-Selected Engineered HAs Elicit Neutralizing Antibody Response in vivo

This example demonstrates that down-selected engineered HAs elicit neutralizing antibody responses in vivo.

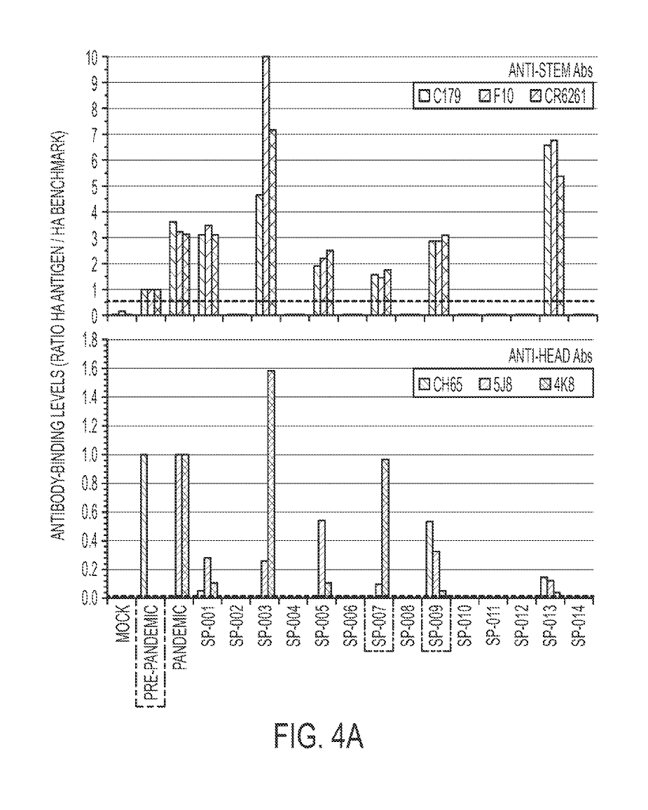

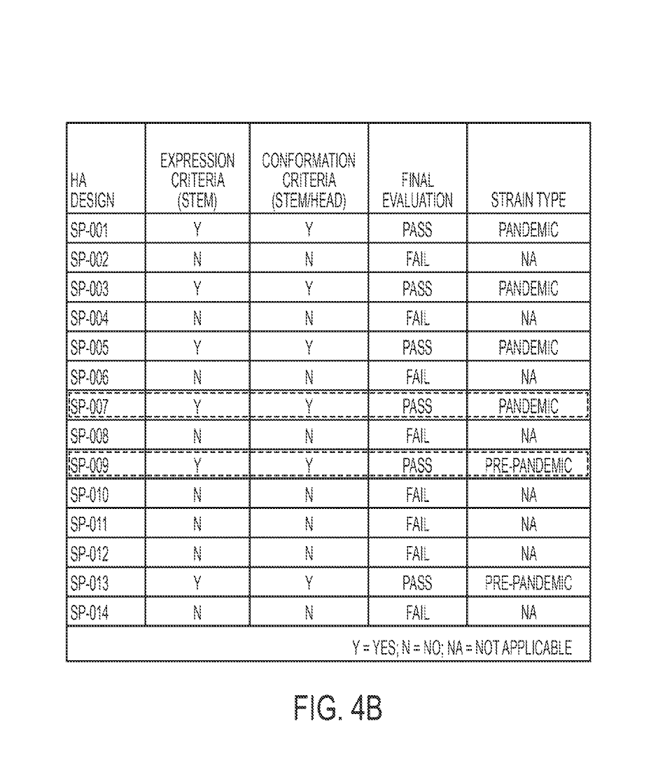

In particular, fourteen engineered HAs were analyzed using the panel of neutralizing antibodies described in Example 2 (anti-stem Abs C179, F10, and CR6261 and anti-head Abs CH65, 5J8, 4K8) and the methods described in Example 1. Exemplary results showing antibody-binding levels are shown in FIG. 4A. Six out of the fourteen met both expression and conformation criteria (see FIG. 4B).

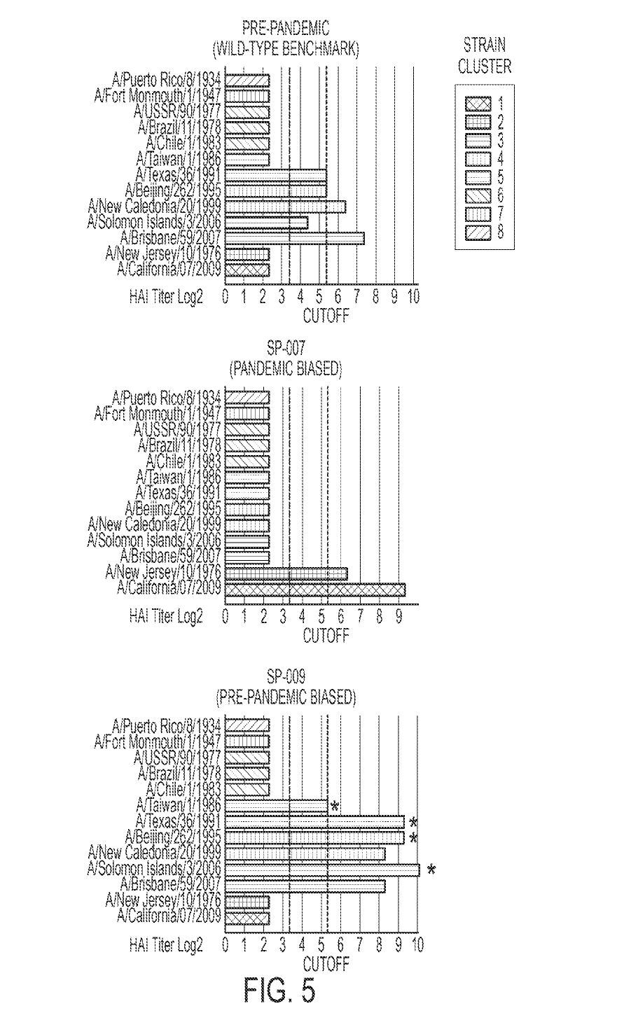

Two of them (SP-007 and SP-009) were selected for in vivo immunogenicity testing. Down-selected HA antigens were produced as part of viral-like particles and used to immunize mice. Wild-type HAs (from pre-pandemic strains) were used as benchmarks. Exemplary results are shown in FIG. 5. Both HA antigens were able to induce neutralizing antibody responses (measured by hemagglutination inhibition assay with immune sera) and showed specificity against strain clusters predicted by anti-head antibody binding analysis. In other words, antibody responses specifically neutralized influenza strains that matched the pattern predicted by our panel of anti-HA head broadly neutralizing antibodies.

In another experiment, twenty-one HAs (70% of all tested HAs) met both expression and conformation criteria. All down-selected HAs retained functional receptor-binding properties in hemadsorption assay and were able to support the production of viral-like particles (VLPs) or live viruses. Immunization with down-selected HAs elicited strong neutralizing antibody responses which recapitulated the neutralization patterns predicted in anti-head bnAb binding assays, further validating approaches to select engineered antigens based on their conformation.

### Example 5

### Successful Incorporation of Screening Assay to Universal Flu Antigen Production Workflow

This example demonstrates that various screening assays described herein have been successfully incorporated to the universal flu antigen production workflow, in particular, as an important part of universal flu antigen evaluation.

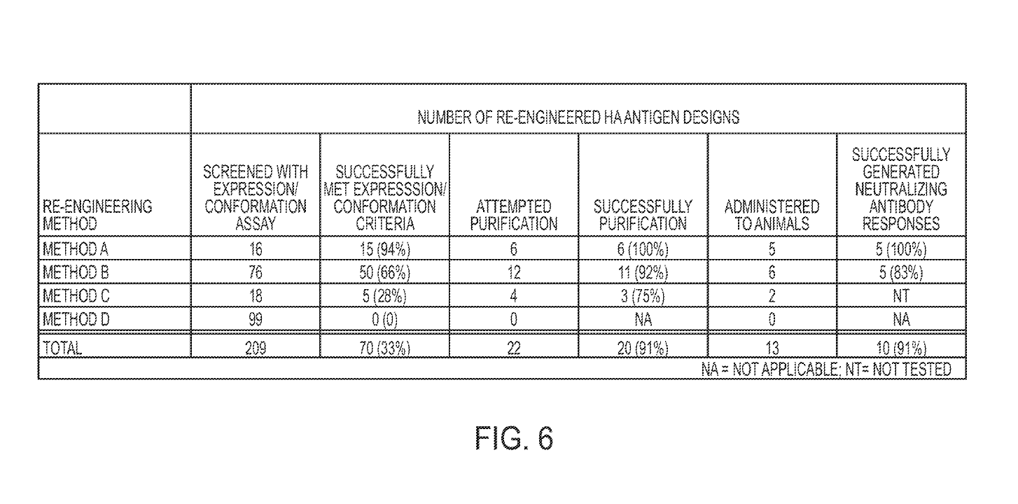

The rapid expression/conformation screening assay has been applied to more than 200 engineered HA antigens (designed by 4 different engineering methods) and one gene library. An exemplary summary is shown in FIG. 6. As shown, out of 209 engineered HA antigens tested, 70 (about 33%) engineered HAs met the expression and conformation criteria. Purification was successfully attempted on 20 out of 22 HAs (about 91%) that met both selection criteria. Thirteen (13) of these purified engineered HAs were administered to animals for in vivo immunogenicity testing. Ten (10) out of thirteen (13) (about 91%) successfully generated neutralizing antibody responses.

This example demonstrates the screening assay described herein have broad application in universal flu vaccine development. It not only validates and identifies rationally designed HA antigens that can produce functional HA proteins, but also provide useful insights into their immunogenicity.

Having thus described several aspects of at least one embodiment of this invention, it is to be appreciated that various alterations, modifications, and improvements will readily be apparent to those skilled in the art. Such alterations, modifications, and improvements are intended to be part of this disclosure, and are intended to be within the spirit and scope of the invention. Accordingly, the foregoing description and drawings are by way of example only and the invention is described in detail by the claims that follow.

### Equivalents

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims.

In the claims articles such as “a”, “an” and “the” may mean one or more than one unless indicated to the contrary or otherwise evident from the context. Thus, for example, reference to “an antibody” includes a plurality of such antibodies, and reference to “the cell” includes reference to one or more cells known to those skilled in the art, and so forth. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are presenting, employed in, or otherwise relevant to a given product or process. Furthermore, it is to be understood that the invention encompasses all variations, combinations, and permutations in which one or more limitation, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for anyone of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is noted that the term “comprising” is intended to be open and permits the inclusion of additional elements or steps.

Where ranges are given, endpoints are included. Furthermore, it is to be understood that unless otherwise indicated or otherwise evident from the context and understand of one of ordinary skill in the art, values that are expressed as ranges can assume any specific value or sub-range within the state ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention can be excluded from any one or more claims, for any reason, whether or not related to the existence of prior art.

The publications discussed above and throughout the text are provided solely for their disclosure prior to the filing date of the present application. Nothing herein is to be construed as an admission that the inventors are not entitled to antedate such disclosure by virtue of prior disclosure.

1. A method of analyzing expression and conformation of engineered hemagglutinin (HA) antigens, comprising steps of
(a) providing one or more cells, each cell comprising a nucleic acid sequence encoding an engineered HA antigen;
(b) immunostaining of the one or more cells with a panel of neutralizing antibodies under conditions that permit the neutralizing antibodies to bind to the engineered HA antigen displayed on surface of the one or more cells, wherein the panel of neutralizing antibodies comprise a plurality of neutralizing antibodies against HA stem and a plurality of neutralizing antibodies against HA head;
(c) detecting binding levels between individual neutralizing antibodies and the engineered HA antigen displayed on the surface of the one or more cells; and
(d) determining if the engineered HA antigen is properly expressed and/or folded based on the binding levels detected between the individual neutralizing antibodies and the engineered HA antigen.

2. (canceled)

3. The method of claim 1, wherein the engineered HA antigen is designed by computational approaches.

4. The method of claim 1, wherein the engineered HA antigen is designed based on consensus sequences among a series of HA proteins from different influenza strains.

5. The method of claim 1, wherein the engineered HA antigen is designed based on the deletion or rearrangement of structural domains.

6. The method of claim 1, wherein the engineered HA antigen is designed based on swap of structural domains derived from multiple influenza strains.

7. The method of claim 1, wherein the engineered HA antigen is rationally designed based on combinations of neutralizing, hemagglutinin B-cell epitope patterns derived from multiple influenza strains.

8. The method of claim 7, wherein the engineered HA antigen comprises cross-reactive epitopes.

9. The method of claim 1, wherein the panel of neutralizing antibodies comprise at least three neutralizing antibodies against HA stem and at least three neutralizing antibodies against HA head.

10. The method of claim 1, wherein the plurality of neutralizing antibodies against HA stem comprise antibodies that bind specifically to one or more conserved epitopes in the stem region of HA from multiple influenza strains.

11-17. (canceled)

18. The method of claim 1, wherein the plurality of neutralizing antibodies against HA head comprise antibodies bind specifically to epitopes within 20 amino acids of the receptor-binding site.

19. The method of claim 18, wherein the epitopes close to the receptor-binding site correspond to the N-terminal end of the short α-helix, site Sa, site Sb, the edge of the receptor pocket, the C-terminus of the short a-helix.

20-25. (canceled)

26. The method of claim 1, wherein the individual neutralizing antibodies or secondary antibodies recognizing the individual neutralizing antibodies are labeled with a detectable entity.

27-28. (canceled)

29. The method of claim 1, wherein the binding levels between individual neutralizing antibodies and the engineered HA antigen displayed on the surface of the one or more cells are detected by flow cytometry.

30. (canceled)

31. The method of claim 1, wherein the method further comprises a step of down-selecting the engineered HA antigen as properly expressed if the binding levels are 50% or greater compared to a wild-type benchmark for at least three neutralizing antibodies against HA stem.

32. The method of claim 31, wherein the wild-type benchmark is defined by the binding levels between the individual neutralizing antibodies and a wild-type HA used for engineering the engineered HA.

33. The method of claim 1, wherein the method further comprises a step of down-selecting the engineered HA antigen as properly folded if the binding levels are over background for at least one neutralizing antibody against HA head and at least three neutralizing antibodies against HA stem.

34. The method of claim 33, wherein the engineered HA antigen is down-selected as properly folded if the binding levels are at least 3 times higher over background for at least one neutralizing antibody against HA head and at least three neutralizing antibodies against HA stem.

35-37. (canceled)

38. An engineered hemagglutinin (HA) antigen down-selected by a method of claim 31.

39. An influenza vaccine comprising an engineered hemagglutinin (HA) antigen down-selected by the method of claim 31.

40-43. (canceled)

44. A kit for analyzing expression and conformation of engineered hemagglutinin (HA) antigens comprising a panel of neutralizing antibodies, wherein the panel of neutralizing antobodies comprise a plurality of neutralizing antibodies against HA stem and a plurality of neutralizing antibodies against HA head.

45-46. (canceled)
